(19) United States
(12) Patent Application Publication
(10) Pub. No.: US 2025/0076685 A1
(43) Pub. Date: Mar. 6, 2025

(54) PORTABLE LIGHT APPARATUS

(71) Applicant: Healyan GmbH, Barchfeld-Immelborn (DE)

(72) Inventor: Philipp CASPARI, Barchfeld-Immelborn (DE)

(21) Appl. No.: 18/459,115

(22) Filed: Aug. 31, 2023

(51) Int. Cl. G02C 11/04

(52) U.S. Cl. CPC G02C 11/04

(57) ABSTRACT

The invention relates to a portable light apparatus configured to be worn by a user, the portable light apparatus comprising a front frame to house eyeglasses and a pair of side frames connected to the front frame, an operating device having at least a battery and at least one electronic part having a light device configured to emit light towards the eyeglasses and/or the user's eyes.

### FIELD OF INVENTION

The invention relates to a portable light apparatus, for example eyeglasses, to be worn by a user.

### BACKGROUND

Sound and stroboscopic, such as light therapy, apparatuses are known which are being used for personal meditation and relaxation, for sleep improvement and visual and neural stimulus as well as medical applications. These range from treatments for psychological disorders like depression, anxiety, post-traumatic stress disorder to neurodegenerative diseases like Alzheimer's disease and Parkinson's disease. The underlying effect for this treatment is so called “brainwave entrainment”.

Further, sound and light therapy has been established as an effective treatment for classic, or winter-based, seasonal affective disorder. Light therapy employs a device which emits significantly more candela than a standard incandescent lamp. Classic “non-stroboscopic” light therapy employs different colors of light to trigger the release of hormones that occur naturally in the human body. Blue light for example increases the release of serotonin and reduces the release of melatonin. Red light however has an inverted effect and additionally increases the production of ATP (Adenosintriphosphat) in skin cells which has anti-inflammatory and anti-aging effect for the skin.

Known light therapy eyeglasses have unflattering appearances with frames that fit to the function of medical applications but lack of features to be worn as normal everyday glasses or sunglasses. The mode of operation for previously available light therapy glasses is to emit continuous light with an increased blue light component for the previously mentioned reasons.

Reading glasses with integrated reading lamps are known. Commonly, these reading lamps are mounted on side frames (also called temples).

### SUMMARY

It is an object of the present invention to provide an improved portable light apparatus, for example glasses, with improved light emitting features, light settings, a combination of the mentioned modes of operation and which has an in appearance such that the portable light apparatus may be worn as normal everyday glasses and/or sunglasses. The provided portable light apparatus of the present invention further allows flexibility in appearance and features, such as exchangeable glasses parts, for a user. Moreover, it is an object of the present invention to combine the features of colored light and strobe light therapy apparatuses such as therapy lamps and reading lamps into wearable glasses with or without visual acuity.

The object according to the portable light apparatus is achieved by the features of claim 1.

Preferred embodiments of the invention are given in the dependent claims.

The portable light apparatus configured to be worn by a user, wherein the portable light apparatus comprises at least a front frame to house a pair of glasses, e.g. eyeglass lenses, and a pair of side frames connected to the front frame, an operating device having at least a battery and at least one electronic part having a light device configured to emit light towards the glasses and/or the user's eyes, wherein the operating device is arranged in at least one of the side frames and/or the front frame, e.g. the battery is arranged in one of the side frames or the front frame and the electronic part with the light device is arranged in the front frame and/or side frames, and wherein the light device comprises at least two light emitting diodes per each of the glasses and the light emitting diodes and/or their emission cones are arranged relative to one another such that the emission cones substantially intersect between a back surface of one of the respective glasses, also just called eyeglass or spectacle lenses, and an apex of the user's cornea to indirectly illuminate a line of sight of the user.

The term glasses means, in particular, eyeglasses or spectacles with or without vision correction, e.g. vision glasses or corrective glasses with lenses for vision correction, glasses or spectacles with or without filters, e.g. sunglasses or glasses protect against radiation, glasses or spectacles with or without safety features, e.g. protective glasses in sports or on constructions, and/or specialized glasses or specialized spectacles, e.g. 3D glasses, fashion glasses, aesthetic glasses.

In other words: the light emitting diodes are arranged with respect to each other such that their emission cones intersect, i.e. overlap at least in an area between the glasses and the user's eyes to indirectly illuminate a vertex distance, in particular at least the full vertex distance. The light emitting diodes may have an illumination direction, e.g. emission direction that is perpendicular to their mounting plane. Alternatively or optionally additionally the emission cones of the light emitting diodes, e.g. light beams of the light emitting diodes, are directed relative to one another such that the emission cones intersect, i.e. overlap at least in an area between the glasses and the user's eyes, in particular between the back surface of one of the respective glasses and the apex of the user's cornea to indirectly illuminate the line of sight of the user, such as the vertex distance. The light emitting diodes may have an illumination direction that is not perpendicular to their mounting plane. “Between the glasses and the user's eyes” means a region between a surface, particular a rear surface, of the front frame facing the user's eyes when worn.

The vertex distance according to DIN EN ISO 13666:2012 is the “distance between the back surface of the lens and the apex of the cornea, measured with the line of sight perpendicular to the plane of the spectacle front”. The line of sight is defined as “line joining the centre of the fovea to the centre of the exit pupil of the eye, and its continuation from the centre of the entrance pupil forward into object space”. The visual point is defined as “point of intersection of the line of sight with the back surface of a lens”. The terms used in this application are based on the basic terms relating to ophthalmic optics, specifically to semi-finished spectacle lens blanks, finished spectacle lenses and fitting purposes according to DIN EN ISO 13666:2012.

It is to be understood that a region in front of the back surface and/or in front of the eyeglasses may be partially illuminated as well.

The advantages achieved by the invention are to provide a user with sufficient indirect light illuminating the line of sight, in particular the area of the visual point of the user. The proposed portable light apparatus (also called portable light therapy apparatus) however may combine as light therapy application at least two modes of light therapy operations, e.g. a first therapy operation also called brainwave entrainment with strobe light and a second therapy operation also called hormone release with colored light. Moreover, the line of sight and the visual point of the user are evenly illuminated in a wider range compared to the state of the art. Additionally the light application can now be activated while a user who relies on eyeglasses as a visual aid is doing everyday tasks. In other words: The invention enables the user to use the glasses in a conventional manner, e.g. as reading glasses, and at the same time to activate a light therapy application. In particular, the glasses keep their original use, e.g. as visual corrective aid, while being equipped with a light therapy application which can be activated for light therapy operation.

The advantage to combine strobe light therapy with regular colored light therapy lies in the flexibility of the modes of operation for the user. Furthermore, altering the color of strobe light may have direct implications on its effectiveness in brainwave entrainment. Because of the local and numerical uneven distribution of retinal cone and rod cells with the highest concentration of cone cells at the fovea, a certain light color may enhance the activation of the cells or dampen it. With the light color being able to be adjusted according to the wish of the user, the brainwave entrainment effect may be fine-tuned. Due to the even illumination distribution of the indirect lighting with multi colored light disclosed in the present invention, all cells across the retina and especially close to the fovea, may be able to be stimulated resulting in a more effective application of light therapy and therefore reducing the required effective treatment time periods significantly.

The visual point is a point of intersection of the line of sight with the back surface of the eyeglass. Visual acuity must be ground such that it is effective in the visual point. Illumination of an area about the visual point may relax the user's eyes. The portable light apparatus may be used as common visual aid glasses and sunglasses. In particular, even with the operating device and the light device a function of common glasses is maintained.

Furthermore, the glasses, e.g. standard glasses, may be equipped with the operating device. The operating device is configured substantially lightweight and compact. The portable light apparatus according to the invention does not require light guide cables. The operating device is easy to install. The operating device comprises at least the battery, the electronic device and the light device. The operating device may be arranged partially or fully in at least one of the side frames. The operating device may be arranged partially or fully in the front frame. The operating device may be arranged partially in at least one of the side frames and partially in the front frame. The battery supplies the electronic device, e.g. a light control unit designed as an integrated circuit, and the light device, e.g. at least two light emitting diodes. At least the battery and the electronic devices may be assembled to form an assembly unit of the operating device which can be releasably attached to the glasses, e.g. to one of the side frames. It is to be understood that the battery and/or the electronic part and/or the light device each or together may be arranged within a space provided anywhere at least in one of the front frame or the side frames, e.g. wherever possible and useful. The light device, e.g. each of the light emitting diodes, may be assembled to form further separate assembly units of the operating device which can be releasably attached to the glasses, e.g. to the lenses.

According to a second embodiment, the portable light apparatus may comprise glasses with reflecting properties, wherein the light device may be arranged such that light is emitted in a predefined angle towards the glasses to reflect towards the user's eyes. The glasses may be configured as light transmitting surfaces. In another configuration of the second embodiment, only parts of the glasses, e.g. lenses, may comprise reflecting properties to direct light towards the user's eye, while other parts remain translucent. The effect of total internal reflection may be used.

A third embodiment relates to a portable light apparatus configured to be worn by a user, wherein the portable light apparatus comprises at least a front frame to house a pair of glasses, e.g. eyeglass lenses, and a pair of side frames connected to the front frame, an operating device having at least a battery, at least an electronic part, e.g. a main electronic part and at least one electronic part, and a light device configured to emit light towards the glasses and/or the user's eyes, wherein the battery and the main electronic part are arranged in at least one of the side frames, the battery is configured to supply energy to the light device via the electronic parts and the electronic part with its light device is arranged in the front frame, wherein the light device comprises at least two light emitting diodes with adjustable emission spectrum assigned per each of the glasses, e.g. per each of the eyeglass lenses, and the light emitting diodes are arranged relative to one another and/or their emission cones are directed relative to one another such that the emission cones substantially intersect between a back surface of one of the respective glasses, e.g. one of the eyeglass lenses, and an apex of the user's cornea to indirectly illuminate a line of sight of the user. In some configurations of the third embodiment, the light emitting diodes may have an illumination direction that is not perpendicular to their mounting plane. In particular each of the light emitting devices (shortly called LED) may be designed as a side view LED. The so called “side view LEDs” may be configured as a LED strip or as regular LEDs coupled with light directing elements, such as miniature lenses for directing emitted light, in particular light beams. Directing emitted light may be achieved by bundling emitted light, in particular light beams, and/or by light bending, e.g. side-bending. Such lenses may be used to direct the emission cones in directions not perpendicular to the mounting plane of the LEDs. A side view LED or light refracting and/or reflecting elements may be used to achieve an overlapping of the LED's light cones. In some configurations, the path of the emitted light of the LEDs may be altered by using light refracting elements such as miniature lenses that are placed on top of each LED, to create an overlap of each light cone. Miniature lenses used for directing light beams and/or emission cones can be made of translucent substrates, e.g. by injection molding. Each miniature lens can comprise a light directing, light bending, light refracting and/or light reflecting structure on at least a portion of the substrate. Via the miniature lenses the emitted light, e.g. light beams, of the LEDs can be aligned at a certain angle relative to one another when mounted. The light beams and/or emission cones of the LEDs can be guided, refracted and/or reflected towards a predetermined area. A combination of both “side view LEDs” and miniature lenses to direct the light may also be a viable configuration.

According to a fourth embodiment, the portable light apparatus may comprise at least one detection device configured to monitor a heart rate, pulse rate, blood pressure, atrial fibrillation and/or other so called “fitness” tracking data. The detection device may be arranged in at least one of the side frames and/or in the front frame which are in contact with the user when worn. Data may be transmitted to a connected external control device such as a mobile device, a smartwatch, a smartphone, a computer or the like. The detection device may be part of the operating device. The detection device may comprise at least one sensor arranged on the main electronic part and/or on the electronic part.

In some designs, the light emitting diodes and/or their emission cones may be arranged, in particular aligned, with respect to each other such that illuminating the area between an inside part of the glasses and the user's eye is ensured in an easy and constructive way independent from head sizes, head forms, nose shapes, eye sockets etc.

As later described, the light device may additionally be used for therapy purposes whereas providing sufficient indirect illumination in the area of the vertex distance may increase effects.

In some designs, the light emitting diodes assigned to one of the glasses may be arranged angled relative to one another such that an intersection area, in particular an overlapping area, of the emission cones is substantially elliptical shaped. In some designs, the emission cones of the light emitting diodes assigned to one of the glasses may be directed at certain angles relative to one another such that an intersection area, in particular an overlapping area, of the emission cones is substantially elliptical shaped. In other words: the emission cones intersect, in particular overlap, each other such that the intersection area is elliptical shaped in side view and/or top view. This allows maximum illumination about and along the line of sight. Unlike state of the art light therapy apparatuses or glasses with reading lamps, the illumination is not provided outside the frame and not just from one direction, for example in front of the front frame and/or towards the side frames.

In some designs, the emission cones, also known as radiation cones or beam pattern, each may have an opening angle or beam angle of less than or equal to 90°. In some designs the emission cones may have an opening angle between 90° and 180°.

In some designs, the light emitting diodes and/or the emission cones may be arranged aligned relative to one another such that an intersection area of their emission cones covers a length range between 5 mm and 35 mm between the back surface of the respective eyeglass and the apex of the user's cornea. For example, a standard distance between the back surface of the eyeglasses and apex of user's eyes, also known as vertex distance, comprises a length of 10 mm to 25 mm, in particular 15 mm to 20 mm. The range of the intersection area covers all possible and standard vertex distances to provide maximum illumination and sufficient brightness within this range.

In some designs, the light device may comprise at least four light emitting diodes assigned to one, in particular to each, of the glasses, e.g. eyeglass lenses. In total, the light device may comprise a minimum of eight light emitting diodes, wherein each glass is illuminated by a minimum of four light emitting diodes. The light emitting diodes and/or the emission cones assigned to each glass may be arranged relative to one another, in particular aligned and/or angled, such that the intersection area of the emission cones is substantially elliptical shaped. In other words: the emission cones intersect each other such that the intersection area is elliptical shaped in side view and/or top view. In front view and cross-section the intersection area is substantially rectangular shaped to cover a large area in relation to the eyeglass.

In some designs, the electronic part, e.g. an auxiliary electronic part and/or a main electronic part, may be configured as a flexible printed circuit board which has an outline shape of the front frame. The electronic part comprising the light device may be configured as a thin, flexible substrate. The electronic part may comprise a number of flexible copper layers. The electronic part may be a printed circuit board with connected LEDs. The electronic part carrying the light device may be arranged between two frame parts, such as a rear part and a front part, of the front frame. The electronic part may be attached, e.g. adhered and/or clipped, to at least one of the frame parts. For example, the electronic part may be sandwiched, clamped and/or inserted between the two frame parts. The electronic part may be a flexible electronic foil in shape of the front frame. The electronic part may be overmolded and/or back-molded by a substrate forming at least one of the frame parts. Due to the thin and flexible configuration of the electronic part with the light device, the light apparatus may maintain its optical appearance as common, wearable eyeglasses compared to other light therapy devices.

The electronic part, e.g. the auxiliary electronic part, may comprise two openings to receive the glasses, wherein the light emitting diodes assigned to one, in particular each, of the glasses are distributed around the openings.

The light emitting diodes may be arranged on a flat or plain surface of the electronic part. The light emitting diodes may have an illumination direction, i.e. light emission direction that is not perpendicular to their mounting plane. In other words: the LEDs may sit on a flat or plain surface of the printed circuit board. To direct the light emission cones of the light emitting diodes at certain angles, in particular into a preferred intersecting and overlapping pattern, to indirectly illuminate the line of sight of the user, light directing elements such as light directing miniature lenses may be coupled to the LEDs, e.g. regular LEDs or side view LEDs. For example, the light directing elements may be arranged on top of the LEDs. Miniature lenses used for directing light beams and/or emission cones can be made of translucent substrates, e.g. by injection molding. Each miniature lens can comprise a light directing, light bending, light refracting and/or light reflecting structure on at least a portion of the substrate. Via the miniature lenses the emitted light, e.g. light beams and/or emission cones, of the LEDs can be aligned at a certain angle relative to one another when mounted. The light beams of the LEDs can be guided, refracted and/or reflected towards a predetermined area. The LEDs with light directing elements may be arranged in corner areas of each opening. This allows an easy alignment and/or orientation of the respective emission cones of the LEDs.

In some designs, a number of projecting tabs arranged in an area of the openings and each carrying one of the light emitting diodes. The tabs may be located in corner areas of each opening. This allows an easy alignment and/or orientation of the respective emission cones of the light emitting diodes.

In some designs, each tab may comprise a tab body having a first end connected to the opening and a second free end extending away from the opening to which the light emitting diode is attached. Each tab may have a bent portion arranged between the first end and the second free end. The tab may be a flap, a lug or the like.

In some designs, each tab may be bent about a bending radius smaller than 5 mm, in particular smaller than 4 mm, preferably smaller than 1 mm. For example, the bending radius may be smaller than 0.8 mm. In an embodiment, the bending radius may be 0.5 mm. Dimensions of the tab are configured to fit into almost any front frame. The bend angle of the tabs may range between 600 and 1200 with respect to the section of the electronic part, e.g. the auxiliary electronic part, that remains plain/flat.

In some designs, each tab may be projecting away from a front surface and projecting towards a back surface of the auxiliary electronic part about an acute angle. In other words: the tabs are protruding from a front or back surface of the auxiliary electronic part. The orientation of the emission cones may be determined and adjusted such that the intersection area is covering the vertex distance. The light emitting diodes may have an illumination direction, e.g. emission direction that is perpendicular to their mounting plane. Thus, arrangement of the light emitting diodes on the tabs allows an easy alignment and/or orientation of the respective emission cones of the light emitting diodes in certain angles.

In some designs, the electronic device, e.g. the auxiliary electronic part, may be made of one-piece and the tabs are shaped out from the opening. The tabs may be formed out, punched out, cut out or molded from the auxiliary electronic part, in particular from the flexible circuit board. Alternatively, the tabs may be attached to the opening of the auxiliary electronic part, for example by welding, soldering, gluing and/or clipping or the like.

In some designs, the electronic part, e.g. the auxiliary electronic part, may comprise at least one bent area in a nose bridge area and a connection area to at least one of the side frames.

In some designs, the battery may be configured to supply energy to the light device via the electronic parts, wherein the electronic part, e.g. the main electronic part, comprises a charging interface, in particular a charging port, to couple with an external charging device to recharge the battery and/or an external control device, for example for remote control of the portable light apparatus. The external control device may be a mobile device, a central server and/or a server-side, for example a so called back-end server, described later.

Emitted light radiation that is allowed to be directed towards the eye or eyeball is researched and defined by norms, for example regarding a maximum threshold of emitted light radiation and safety implications for the eyes regarding light sources, such as LED light sources, emitting visible optical radiation. Light sources and LEDs are consequently evaluated and categorized in terms of optical safety in accordance with the photobiological safety standards for different systems: IEC 62471 (International)/EN 62471 (EU) and ANSI/IESNA RP-27(USA).

Care is taken to design the portable light apparatus such that it falls within these standards.

Further, these standards define a number of different Risk Groups (RGs) for light sources and LEDs. It is an object of the present invention to design the portable light apparatus such that it ensures safety to the user with no hazard, for example, no photobiological hazard, on exposure. These properties are defined, for example, by Risk Groups RG0 and RG1.

The light device is arranged in the front frame such that light emitted by the light device is directed towards the user's eye. There is no need for a reflection of the light off of the actual eyeglasses.

Light emitted by the light device may be guided along a shape of the front frame, in particular in an environment of the eyeglasses. Therefore, providing specific reflection elements to reflect the emitted light by the light device are not necessary. The emitted light is directly directed towards the user's eye.

In some designs, the portable light apparatus is provided as light therapy apparatus, such as light therapy glasses to be worn by a user. The portable light apparatus may be provided as stroboscopic light therapy glasses. The portable light apparatus may be configured as regular glasses and/or sunglasses and/or reading glasses to be worn by a user.

The portable light apparatus has the advantage to have the appearance of a regular looking glasses, such as reading glasses, sunglasses and/or design glasses, whilst providing features of therapeutic and/or medical light applications, such as for personal meditation and relaxation, at any time the user is in need.

In some designs, the portable light apparatus is also suited for use to increase a concentration level and/or focus level, for example during work, sports, driving or any other activity.

The portable light apparatus, in particular light glasses, may also provide a fun factor to the user who may stand out with the apparatus when the light device is on, for example at events.

The user is not forced to exchange regular glasses with a portable light therapy device for therapeutic and/or medical light applications which reduces effort and time and make it simpler to use for the user. Therefore, the portable light apparatus according to the invention may also advantageously increase a frequency of use for the user. In some designs, for example, the portable light therapy device may comprise prescription lens and therefore additionally serve as both prescription glasses. In some designs, prescription lens in such a portable light therapy device may be photochromic, for example become darkened or colored under sunlight or high-intensity light. In some designs, the portable light apparatus has may comprise sunglasses, which may, for example, serve as a protective eyewear designed to prevent bright light from damaging or discomforting the eyes, for example by at least partially blocking UV radiation. The sunglasses may also reduce undesired distractions during the application of the disclosed portable light therapy device. In some designs, the light therapy device may comprise polarized glasses. In some designs, the glasses may additionally comprise blinders, for example to further minimize distractions during use. Such blinders may be attached either to the outer or to the inner side of the glasses.

The front frame and/or side frames, also known as temples, may have a shape, form, design and/or color and/or pattern desired by a fashion designer, a customer and/or a user.

In some designs, the operating device may be configured to be adaptable to regular looking glasses frames and side frames. For example, in some configurations, the spectacle manufacturers, sellers and/or customers, may be able to easily retrofit existing spectacles and/or spectacle frames with the disclosed portable light operating device and/or adapt to the portable light operating device.

As disclosed, the eyeglasses for the portable light apparatus may be configured as regular transparent or colored glasses, ophthalmic grade glasses or lenses, color tinted glasses and/or functionally coated glasses. In some designs the eyeglasses may comprise anti glare, UV filters, polarisation filters, thermochromic or electrochromic coatings or any other coating and/or combination of all above. For clarity, the eyeglasses may have lenses with or without light attenuating function and/or visual acuity, in particular eyesight.

Advantages of electrochromic coated glasses may consist of providing different properties to the user such as lightly tinted glasses, fading properties, different colors which may be adjusted by means of a input device on the portable light apparatus and/or via a user interface on the mobile device connectable to the portable light apparatus.

Furthermore, the electrochromic coated glasses may provide a so called shutter effect which may be adjustable. Electrochromic coatings may become dark when a voltage is applied and/or when charges are added to an optical part of the coating. When voltage and/or charges are removed, the coating may become transparent.

In some designs, it may be preferable for a total weight the portable light apparatus, with and/or without eyeglasses, to be in a certain range. Too heavy weight may induce discomfort and undesirably reduce the device usage. For example, in some designs, the total weight of the apparatus may preferably range from about 5 g to about 200 g, for example 10 g to 150 g. In some designs, the total weight of the portable light apparatus may range from about 20 g to 100 g to be in the most comfortable range for the user to apply it regularly.

In some designs, the auxiliary electronic part may be a printed circuit board (PCB). In some designs, the auxiliary electronic part may be a flexible printed circuit board (flexible PCB, FPCB) which has an outline shape of the front frame. The auxiliary electronic part may be configured as a flexible cable or a flexible plate.

In some designs, the auxiliary electronic part may be a purely flexible PCB or a rigid PCB. The auxiliary electronic part may consist of at least one rigid PCB and at least one flexible PCB. The auxiliary electronic part may consist o two rigid PCBs connected by at least one flexible PCB. The auxiliary electronic part may consist of at least two flexible PCBs connected by at least one flexible or rigid PCB. The PCBs of the auxiliary electronic part may be connected to each other by a number of cables, such as individual cables or in form of a ribbon cable. Therefore, a stable connection between the PCBs of the auxiliary electronic part may be easily achieved.

In some designs, a connection between the main electronic part and the auxiliary electronic part may be connected to each other by a number of cables, for example a number of individual cables or in form of a ribbon cable. Therefore, a stable connection between the main electronic part and the auxiliary electronic part in a connection area of the front frame to the side frame may be easily achieved.

In some designs, the auxiliary electronic part may be configured reversibly flexible and/or elastic. The auxiliary electronic part may be a reversibly flexible and/or elastic PCB. Thereby, the auxiliary electronic part may be formed adaptable or designed to fit the front frame, for example to movements and/or bends of the front frame and/or the side frame. Conductive paths may be configured meander-shaped or the like, providing flexible and/or elastic and/or bending properties.

In some designs, the auxiliary electronic part may be configured stretchable. The auxiliary electronic part may be a stretchable PCB. The auxiliary electronic part may be made of thermoplastic polyurethane. Conductive paths may be configured meander-shaped or the like, providing flexible and/or elastic and/or bending properties.

The auxiliary electronic part may comprise a shape with openings, for example cutouts, of the shape of the eyeglasses to not block the user's vision through the eyeglasses held by the front frame. In some designs, the auxiliary electronic part may be substantially plate-shaped and comprises at least one cutout in the shape of the front frame.

In some designs, the auxiliary electronic part may be substantially circular-shaped or semicircular-shaped or square-shaped or may comprise any other shape. The auxiliary electronic part may be configured to follow an outline of the front frame. The auxiliary electronic part may comprise two openings, for example cutouts, one for a left eyeglass and one for the right eyeglass. In another embodiment, two auxiliary electronic parts may be used, each one for each eyeglass area in the front frame.

In some designs, connector elements, such as connector pins, contact pins or electrical connectors or electrical pins and corresponding connector sockets or contact holes, may be used in the connection area between the front frame and at least one of the side frame providing a releasable connection of the main electronic part and the auxiliary electronic part. The connector elements may be spring-loaded.

One end of the main electronic part, which is arranged in the at least one side frame, may comprise a number of first connector elements, facing towards the front frame. One end of the auxiliary electronic part, which is arranged in the front frame, may comprise a number of second connector elements corresponding to the first connector elements, facing towards the at least one side frame. In a use state of the portable light apparatus to be worn by the user, the connector elements are engaged, wherein the main electronic part and the auxiliary electronic part are in electrical connection. In a non-use state or stowed state of the portable light apparatus, the connector elements are disengaged. Alternatively or optionally additionally, the electrical connection interface, releasable or non-releasable, for example temporary or permanent connections, is provided by a number of cables and/or other connector elements, such as plug blades.

A suitable bend radius of the auxiliary electronic part may be in a range of a few millimeteres. A bend radius may be selected depending on a total thickness of the printed circuit board. In some designs, the bend radius may range from about 0.1 mm or greater to any needed and/or desired value, and direct emitted light by the light device towards the user's eye. In some designs, the auxiliary electronic part, in particular in form of a flexible printed circuit board, between the front frame and the corresponding side frame may be bent about a predefined radius of a few millimeters.

The bend radius at the connection area, in particular connection interface part, of the frame part to at least one of the side frames may be in a range of a few millimeters. In some designs, the bend radius may range from 0.1 mm or greater to any needed and/or desired value.

The flexible auxiliary electronic part may comprise at least one stiffener layer located at least in an area underneath the light device, in particular underneath each light emitting diode (LED) of the light device. The light device comprises at least one light emitting diode (LED) soldered or otherwise attached thereto.

The maximum light intensity (total radiance of the light device—radiance being the radiant flux per unit solid angle per unit projected area) of all of the LEDs in the portable light apparatus combined may preferably range from about 0.0001 W/(m2*sr) to about 4*106 W/(m2*sr) for electromagnetic radiation in the wavelength range of 150 nm to 3000 nm. Too low intensity may reduce the impact of the device, while to high intensity may induce discomfort or various undesirable side effects. The maximum light intensity of the individual LED may also preferably range from about 0.0005 W/(m2*sr) to about 0.001 W/(m2*sr). Too high intensity of the individual LED may similarly lead to discomfort or side effects.

The total number of the LEDs may range from 1 to about 1000,000. The larger number of LEDs may enable a smoother effect for the user because a more evenly distributed illumination of the area of the user's eye can be achieved, but often at the expense of additional cost or complexity. The suitable size of the LEDs footprint may range from about 1 μm2 to about 100 mm2.

In some designs, the spacing between the individual LEDs may range from about 0.01 μm to about 100 mm.

Examples of suitable LEDs include but are not limited to the standard package sizes referred to as: 0204, 0603, 0805. These standardized sizes allow for a reduction in production cost of the portable light apparatus, while being large enough to output enough light to create the desired effect.

In some designs the LEDs can be arranged in an array that utilizes organic light emitting diodes (OLEDs), forming one surface that can appear to the user's eye coherent, so that the user is unable to identify a single LED or OLED with the naked eye. One example of such an array may commonly be referred to as an OLED display. The application of an OLED display into the portable light apparatus would benefit the freedom of design.

In some designs LEDs with a di-, tri, tetra- or multichromatic spectrum may increase the luminous efficacy of the portable light apparatus. In other designs LEDs with a variable wavelength output spectrum might be utilized. Packages that include RGB LEDs may be utilized to enhance certain stimuli for the user according to their wishes. As an example, the increased output of blue light can lead to an increased output of the hormone and neurotransmitter Serotonin, which in return may help the user waking up in the morning.

The light emitting diodes may utilize but are not limited to the following base materials and Elements to radiate electromagnetic waves in the wavelength range of 150 nm to 3000 nm: GaN—Galium Nitride, InGaN—Indium Galium Nitride, Saphire, ZnSe—Zinc Selenide, Si—Silicone, InGaN—Indium Galium Nitride, P—Phosphorous, YAG—doped Yttrium Aluminium Garnet, AlGaN—Aluminium Galium Nitride, AlGaInN—Aluminium Galium Indium Nitride, BN—Boron Nitride, organic compounds like organic molecules in a crystalline phase including polymers.

In some designs the light output of the LEDs may be altered by the package the LED is manufactured with. One example of this may be a polymer lens that alters the output spectrum and directs the light into a desired direction. Also reflective layers within the LEDs package may be utilized to direct the light towards the user.

In some designs, the LED may be arranged in a cutout, recess, opening or the like provided in the auxiliary electronic part. The LED, for example, may comprise one end which is soldered or otherwise attached to the auxiliary electronic part, for example to a region which has a bend, and another end which is arranged in the cutout.

In some designs, a stiffener layer may be located on the auxiliary electronic part at least in an area underneath, above or both at the bent area of the connection area to at least one of the side frames.

In some designs low powered LASERs with the output range of 0.0001 W/(m2*sr) to about 4*106 W/(m2*sr) may be utilized. The emitted monochromatic coherent radiation can be directed towards the user's eye utilizing no lenses, a collimating lens, or multiple lenses to form the beam in a useful manner to create the desired effect of the portable light apparatus.

In some designs, a stiffener layer may be located on the auxiliary electronic part at least in a connection area of the auxiliary electronic part to a main electronic part, such as a main printed circuit board (PCB) which will be specified later, wherein the main electronic part is arranged in at least one of the side frames. This area corresponds to the connection area of the front frame to at least one of the side frames.

In some designs, the front frame may comprise two frame parts permanently or temporarily connected to one another to hold the eyeglasses. In one configuration, the front frame parts are connected to each other in a material-locking manner, force-locking manner and/or form-locking manner.

In some designs, the magnet may exhibit one or more of the following compositions: NdFeB—Neodymium Iron Boron, AlNiCo—Aluminium Nickel Chromium, SmCo—Samarium Cobalt, BiMo—Bismuth Mangan, Ferrite and other Iron based alloys. The “strength”—also referred to as maximum energy product, meaning the energy density of the individual magnet may range from about 10 kJ/m3 to about 3000 kJ/m3. The size of the individual magnets may range from a volume of about 1 mm3 to about 1000 mm3.

The excessive magnet size may undesirably increase weight or aesthetics. The excessive magnet strength may similarly be uncomfortable, and create electromagnetic interference, while too low strength may reduce the functionality of the joining technique.

In some designs, the frame parts may be joined together with screws, magnets, snap fittings, glue, friction welding, overmolding or any other temporarily or permanent joining technique.

In some designs, if the front frame parts may be temporarily connected to each other via a force-locking manner, also known as force-fitting manner, the eyeglasses may be exchanged by separating the frame parts. In some designs, such temporary connections may enhance the operational flexibility and provide superior user experience.

In some designs, the separate pieces, namely the frame parts, may be configured to hold the eyeglasses and the auxiliary electronic part with its light device. The frame parts may be manufactured, for example, by using injection molding, additive manufacturing, vacuum forming, milling or any other manufacturing method.

In some designs, in a detached state of the front frame, in particular the frame parts, the eyeglasses may be exchangeable and wherein the auxiliary electronic part and the light device are fixedly arranged in one of the frame parts. The auxiliary electronic part with its light device is held in place by one of the frame parts.

In some designs, the front frame is mechanically connected to the side frames, for example via a hinge joint or any other hinge and/or joint mechanism. The side frames are foldable in a direction towards and away from the front frame. In some designs, one of the frame parts is connected to the side frames via a respective hinge joint.

In some designs, the at least one side frame comprising the main electronic part may be connected to the front frame via a connection mechanism with more than one degree of freedom. The connection mechanism may be configured as a hinge and slide mechanism, for example a rotary-sliding hinge. In some designs, a pivot joint is configured as a sliding joint at the same time, wherein pivot movement of the side frame relative to the front frame, when folding from the use state to the non-use state of glasses, causes the side frame to slide relative to the front frame. Not only a user experience may be improved, but maintaining a permanent electrical connection between the main electronic part and the auxiliary electronic part or connecting and disconnecting a releasable electrical connection between the electronic parts may be simplified. Furthermore, the connection mechanism with more than one degree of freedom may compensate different head sizes as well as reducing a distance between the side frame and the front frame so as to reduce folding and/or bending of an electrical connection interface between the main electronic part and the auxiliary electronic part.

In some designs, at least one of the side frames may hold one or more batteries and the main electronic part of the operating device. In some designs, at least one of the side frames may comprise a mechanical power switch to connect a circuit of the main electronic part to the at least one battery. In some designs, at least one of the side frames may comprise a charging port to recharge the one or more batteries, for example such as lithium-ion batteries, from an external charging device, in particular an external power source.

In some designs, the at least one side frame may hold lithium-ion or lithium metal batteries, such as pouch-type, coin-type, cylindrical-type or prismatic-type batteries, and the main electronic part. The use of lithium-ion or lithium metal batteries may enable high specific energy, long cycle life, long calendar life and other advantages often lacking in other battery chemistries. In some designs, the cathode in such batteries may preferably comprise layered lithium cobalt oxide (LCO) or lithium nickel cobalt manganese oxide (NCM) to enable sufficiently high energy density.

In some designs, the operating device may comprise at least one main electronic part, namely the main printed circuit board, which may be arranged in one of the side frames having a number of interfaces, wherein a first interface providing a connection to the auxiliary electronic part, a second interface providing a connection to the battery and at least a third interface providing connection to an external charging device.

In some designs, the main electronic part may hold the first interface, in particular a communication interface, that permanently or temporarily connects the main electronic part located in one of the side frames to the flexible auxiliary electronic part located in the front frame. The main electronic part may hold the second interface, for example a power line, that permanently or temporarily connects the main electronic part to the battery. The main electronic part may hold the third interface, for example a communication interface, that is able to connect to the external charging device when, for example, the portable light apparatus is coupled to a charging device. The portable light apparatus may be charged wirelessly or wired. The wireless charging may be preferable in some designs, as it may enhance user experience, because classic wired solutions like cables and contact pins are prone to wear and degradation over time the wireless charging capability might increase the lifespan of the portable light apparatus.

In some designs, the operating device may comprise at least a processor module, a light device driver and a voltage regulation circuit and battery charging circuit, a charging connector and/or a power switch. The main electronic part may comprise the processor module, the light device driver and the voltage regulation circuit and battery charging circuit, the charging connector and/or the power switch as electronic components.

The main electronic part may hold a communication chip with at least one or more processing cores, for example configured as the processor module, and a radio module, such as a communication interface, to transmit and exchange data using radio waves. In some designs, the radio module may be configured to transmit and exchange data using low energy. This is, for example, known from audio streaming over radio waves. The radio module is configured to communicate via audio data. The radio module is configured to communicate over a low energy connection with reduced power consumption.

In some designs, the radio module may be configured to communicate over a so called Bluetooth Low Energy (BLE) connection. BLE is a wireless network technology standard intended to provide considerably reduced power consumption while maintaining a defined communication range. BLE is configured to exchange data over a defined distance between Bluetooth-compatible devices, including fixed and mobile devices.

In some designs, the operating device may comprise a light device driver to control stroboscopic frequency and/or light sequences of the light device in the front frame. The main electronic part may hold a multi-channel driver chip, for example configured as the light device driver, to receive logic level signals, for example between 0.1 and 100 Hz, from the communication chip to control stroboscopic frequency, for example for generating a light sequence, of the light device in the front frame. Further, the stroboscopic frequency may be adjusted. In some designs, adjustment steps may be about 0.01 Hz for a “smooth experience”.

In some designs, the main electronic part may hold a power transistor, such as a single power transistor, to receive a signal, such as a pulse-width-modulation signal, from the communication chip to control a power level, in particular energy level, of the light device in the front frame.

In some designs, the main electronic part may hold the voltage regulation circuit to power, that means to supply energy to the communication chip and the light device on the auxiliary electronic part.

Further, the main electronic part may hold the battery charging circuit and a charging controller circuit to recharge the at least one battery located in at least one of the side frames.

In some designs, the operating device comprises a communication interface providing connection to a mobile device, a central server and/or a back-end device to exchange user data. The main electronic part may hold the communication interface. The communication interface may be provided to remotely control the operating device from the mobile device and/or the central server and/or the server-side device.

The operating device may communicate with the mobile device and/or the central server to analyze, for example, played audio files on the mobile device and/or the central server and, based on user settings, to synchronize and/or generate an appropriate light frequency and/or light sequence of the light device according to the audio listened by the user. A combination of audio listened by the user and light effects applied to the user has been surprisingly effective for therapy.

In summary: It is object of the invention to miniaturize and integrate electronics to an operating device so it may be put into regular looking glasses frames and temples. The stroboscopic light effects will be generated by direct or indirect light sources, for example coming from suitable light emitting diodes (LEDs) that sit inside the front frame part so vision of the user is not blocked and the glasses may be worn as normal everyday glasses or sunglasses. The glasses may be equipped with tinted glasses or ophthalmic grade lenses. To change and integrate the glasses and/or lenses of choice inside the front frame, it may be split up into two frame parts so the glasses/lenses may be placed in one frame half and the other half is placed on top, to form a cavity around the glasses/lenses circumference to hold the glasses/lenses in place. To achieve a fashionable, modern and neutral look the LEDs may be supplied with electrical energy using minimal space inside the front frame for supply lines. Because of its needed size, the battery to supply the LEDs with electrical power has to be located in the temple. To electrically connect the components inside the temple and the flexible PCB in the front frame, spring loaded pins may be used and opposing target connector surfaces. In another example to electrically connect the components inside the temple and the flexible PCB in the front frame a flexible ribbon connector cable may be used between the two PCBs, namely the main printed circuit board and the auxiliary printed circuit board. In another example to electrically connect the components inside the temple and the flexible PCB in the front frame a permanent flexible connection piece may be integrated into the flexible LED—PCBs side. The circuitry integrated into the portable light apparatus comprises a charging system for the battery, such as a common Li-battery, the main processing chip module with integrated antenna, the LEDs and LED-driver chips as power regulators, and a voltage regulation system supplying the main processor, LED drivers and LEDs. The portable light apparatus has a power switch, so all the electrical components except the charging circuitry may be permanently disconnected from the battery supplying them. On the same temple, a pair of charging target connectors may be located so the portable light apparatus may be charged with spring loaded connectors when they are placed inside a dedicated charging device, such as a charging case as introduced later. Alternatively or optionally additionally, the portable light apparatus may be charged via a charging cable with or without the case. Furthermore, the portable light apparatus may be equipped with a magnetic charging device, for example such as a magnetic snap or clamp device to interact with a magnetic charging cable.

A charging case according for the portable light apparatus may be provided and may comprise at least a housing having a cavity in which the portable light apparatus is insertable to be held and protected, and a charging operating device arranged in the housing, wherein the charging operating device is configured to charge a battery of the portable light apparatus when inserted in the cavity.

In some designs, when connected to the charging case, the portable light device may transmit stored data comprising measured information about the user's health and fitness status. The operating device may comprise a storage unit configured to store data detected by the detection device. The stored data may comprise information about a heart rate and/or pulse rate etc. The charging case may be coupled to an external device, such as a computer, a laptop and/or a smartphone.

In some designs, the charging case may comprise at least one battery, for example a rechargeable battery, to charge the at least one battery of the portable light apparatus.

The charging case has the advantage to be portable and to provide storage and protection for the portable light apparatus, in particular in form of glasses. The user may use the charging case as a regular storage and protection case. The charging case enables the user to recharge the portable light apparatus in a simple and comfortable manner. The charging case may have the appearance of a regular glasses case.

The charging case may comprise at least one compartment for storing a number of different exchangeable eyeglasses for the portable light apparatus. In some designs, the housing may comprise a separate compartment below or above the cavity in which the portable light apparatus is insertable. Therefore, the user carrying the charging case may adapt the portable light apparatus to different needs and purposes. The portable light apparatus may be used as regular glasses or sunglasses.

In some designs, the portable light apparatus configured to be worn by a user may comprise a front frame to house eyeglasses and a pair of side frames connected to the front frame, an operating device having at least a battery, at least one auxiliary electronic part having a light device configured to emit light towards the eyeglasses and/or the user's eyes, wherein the battery may be configured to supply energy to the light device via the auxiliary electronic part, wherein the operating device is partially or completely arranged in one of the side frames and/or in the front frame. In some designs, the battery may be arranged in one of the side frames and the auxiliary electronic part and the light device may be arranged in the front frame and/or one of the side frames.

The portable light apparatus may comprise any feature of described embodiments and combination of features as described above.

In some designs, the housing, in particular a shell, of the charging case may comprise two corresponding housing parts, wherein one of the housing parts forms the cavity and the other one of the housing parts is configured as a lid to close or open the cavity. The housing parts may be connected to each other by a hinge joint to form the cavity that may be opened and closed.

In some designs, the housing may comprise a separation line between the two housing parts. A notch may be located at the separation line.

In some designs, the housing parts may contain magnets that are oriented to attract the respective parts. The cavity may be formed by the hinge-connected housing parts. The cavity cannot open on its own while the charging case is oriented in any direction.

In some designs, the cavity may be formed such that an insert direction of the portable light apparatus is defined for the user. The cavity may be shaped such that the portable light apparatus is tightly inserted and fitted when arranged in the cavity. The specified insert direction may refer to a specified folding configuration of the side frames.

In some designs, at least one of the housing parts may comprise a holding element configured to guide the user to insert and place the light apparatus and to removably hold the portable light apparatus in place in the cavity. The cavity, in particular an opening of the cavity, may be oriented in one direction. The cavity may be upholstered.

In some designs, the holding element may be a magnet element. The holding element may be configured to guide the user to place the portable light apparatus into the cavity and to hold the portable light apparatus in place.

The housing parts may be manufactured using injection molding, additive manufacturing, vacuum forming, milling or any other suitable manufacturing method.

Additional pieces to the housing parts may be permanently or temporarily joined with screws, magnets, snap fittings, glue, friction welding, overmolding or any other temporary or permanent joining techniques.

In some designs, the housing comprises at least one charging port to establish a temporary electrical connection to the portable light apparatus.

In some designs, at least one of the housing parts may hold charging electronics for charging the battery of the portable light apparatus. At least one of the housing parts may hold a corresponding counterpart to a charging interface, in particular a charging port, of the portable light apparatus to establish a temporary electrical connection.

Further, the housing, for example one of the housing parts, may comprise at least one display device to display a battery status of the charging case and/or the portable light apparatus. The display device may be configured to give the user a visual feedback about a charging status of the portable light apparatus and/or of a battery of the charging case itself. The charging case may, for example, be rechargeable. The charging case may comprise a charging interface to connect to an external charging device. The charging case may comprise a battery, for example a lithium-ion battery. In some designs, one of the housing parts may comprise at least one cutout for the display device and/or another electrical element that displays the battery status.

In some designs, the charging electronics of the charging case may comprise at least one printed circuit board (PCB). The charging electronics may comprise at least one rechargeable battery. The charging electronics may comprise a charging circuit accessible wired, for example via a so called Universal Serial Bus (USB), to recharge the at least one battery of the charging case. The charging electronics may comprise at least one voltage level regulating circuit to provide correct voltage and power to recharge the battery of the charging case. The charging electronics may comprise a charging port that connects to a corresponding port of the portable light apparatus. Further, the charging electronics may comprise the display device, for example an indicator element, to visually display the battery level of the at least one battery inside the charging case.

In summary: The charging case may contain a battery and PCB circuitry, to be charged using a USB-Type-Standard. The voltage of the battery inside the charging case may be stepped up to a standard, for example, a standard 5V USB voltage level and supplied to the spring loaded pins located inside the case, so it may charge the portable light apparatus when they are connected by placing them inside the charging case. The positioning of the portable light apparatus in the charging case may be taken care of, by placing, for example, permanent magnets inside the frames, such as the front frame and/or the temples of the portable light apparatus, and the charging case that are configured to attract each other.

The control system according to the invention providing remote control for a portable light apparatus to be worn by a user, may comprise at least a light sequencing algorithm and a communication interface providing connection to a mobile device and/or a central server, wherein depending on data transmitted from the mobile device and/or the central server to the communication interface the light sequencing algorithm generates a light sequence for the portable light apparatus.

The control system has the advantage to be compact in design and so as to be easily integrated into a glasses frame design as well as providing reliable wireless or wired connection to the mobile device and/or a central server to exchange data.

The control system may further allow remote control for the portable light apparatus, such as glasses, so that the portable light apparatus may be controlled from remote devices to allow a simple user interface. Furthermore, the control system may be configured to enable user engagement with the portable light apparatus and social engagement between individual customers for example on a centralized platform for their own portable light apparatus which will be described later.

The control system may be configured as a processor module integrated in the portable light apparatus, for example to an operating device, in particular to a main printed circuit board (PCB) which runs the portable light apparatus. The control system may be software provided on the operating device, in particular on the main PCB, of the portable light apparatus.

The portable light apparatus configured to be worn by a user may comprise a front frame to house eyeglasses and a pair of side frames connected to the front frame, an operating device having at least a battery, at least one auxiliary electronic part having a light device configured to emit light towards the eyeglasses and/or the user's eyes, wherein the battery is configured to supply energy to the light device via the auxiliary electronic part, wherein the operating device is partially or completely arranged in one of the side frames and/or in the front frame. For example, the battery may be arranged in one of the side frames and the auxiliary electronic part and the light device are arranged in the front frame and/or one of the side frames. The portable light apparatus may comprise any feature of embodiments and combination of features as described above.

In some designs, the operating device may comprise at least one main electronic part, namely the main printed circuit board, which may be arranged in one of the side frames having a number of interfaces, wherein a first interface providing a connection to the auxiliary electronic part and a second interface providing a connection to the battery.

The main electronic part may hold a communication chip with at least one or more processing cores, for example configured as the processor module, and a radio module, such as a communication interface, to transmit and exchange data using radio waves. In some designs, the radio module may be configured to transmit and exchange data using low energy. This is, for example, known from audio streaming over radio waves.

In some designs, the control system may be running on the communication chip of the portable light apparatus or on a separate chip not yet integrated to an apparatus.

In some designs, the control system may be configured to be responsible to process at least one of the following described tasks. The control system may be configured to receive audio data from an external source, such as the mobile device and/or the central server, via radio waves. The control system may be configured to transmit the received audio data to an audio amplifier or audio output device, for example a loudspeaker or bone sound transducer. The control system may be configured to share or forward data with devices that use the same radio wave communication technology. Examples for other devices may be: transcutaneous electrical nerve stimulation device and/or an electric actuator creating oscillations that may be felt by the user via touch. Furthermore, for example, piezo actuators and/or an electric motor can be used to create oscillations that may be felt by the user via touch and/or have other effects other than being felt via touch.

The control system may be configured to manage over software updates, wireless or wired. The control system may be configured to retrieve, collect and exchange user data with the external source based on user settings. The control system may be configured to store user input variables necessary to generate light sequences of the light device of the portable light apparatus. The control system may comprise a user authorization and activation based on input data from the mobile device and/or the central server, for example from an application and/or client-side, and/or from a server-side, for example a server-side database or a so called back-end server where data and source codes may be programmed, updated, managed and stored.

The control system may be configured to receive measurements of bio sensors. The control system may comprise a bio sensor feedback integration that takes live sensor measurement data into account to fine-tune the light sequences of the portable light apparatus to further enhance the user experience. Those Sensors may be, but are not limited to: heartrate sensor, blood pressure sensor, blood oxygen sensor, EEG-electroencephalography measurement data and fMRI—functional magnetic resonance imaging data.

In some designs, the control system may communicate with the mobile device and/or the central server to analyze, for example, played audio files on the mobile device and/or the central server and, based on user settings, to advantageously, for example for a more favourable user experience and enhanced affect, synchronize and/or generate an appropriate light frequency and/or light sequence of the light device according to the audio listened by the user. A combination of audio listened by the user and light effects applied to the user has been surprisingly effective for therapy.

The mobile device may be a smartphone or a tablet or a smartwatch or the like. The central server may be provided by an application (so called APP) on a computer and/or mobile device. The central server or client-side may run on a user's device through which user interface the user may interact with the portable light apparatus and/or server-side.

The application, such as a smartphone application, also known as APP (short), may be adapted to run on different operating systems of mobile devices.

Graphical details of a user interface may vary on each of those operating systems. The user interface may consist of at least four sections, such as “Home”, “Search”, “Sequence” and “Account”.

In some designs, in the section “Home” the user may have the ability to engage in at least the following interactions: see a timeframe based overview of personal use of different frequency bands at least daily, weekly, monthly and annually; manage own sequenced audio tracks, playlists and albums as well as those of other users they follow which are made public by those users; interact, such as known “like” and “comment” interactions, with news feed filled with at least latest tracks, playlists, albums and posts based on users they follow.

In some designs, in the section “Search” the user may have the ability to engage in at least the following interactions: search specifically at least for publicly available tracks, playlists, albums and other publically shared user profiles; see suggested music titles and/or audio titles, for example audio book titles and audio track titles, based on genres from at least one and not limited to known platforms and/or providers; see a set of suggested sequencing settings, in particular light frequency and/or sequence settings, for each of the music and/or audio files.

In some designs, in the section “Sequence” the user may have the ability to engage in at least the following interactions: create their own set of individual sequencing settings for personal use and/or shared use; transmit those settings to their own portable light apparatus, store the settings in the application and/or in the server side database; link their chosen sequencing settings to music and/or audio tracks, playlists and/or albums; create a set of different settings that are linked to music and/or audio tracks, playlists and/or albums; create a set of different settings that a linked to music and/or audio tracks, playlists and/or albums with dedicated comments and recommendations of use.

In some designs, in the section “Account” the user may have the ability to engage in at least the following interactions: activate their portable light apparatus; manage their subscription; manage profile settings at least for privacy and social interaction.

The application may advantageously have an application programming interface (API) which is configured to exchange data with the server side database at least but not limited to the following: user account and payment info; user sequencing settings data; duration of use; type of use.

The API may be configured to exchange data also with APIs from third party streaming providers from at least one and not limited to platforms and/or providers.

The server-side database may consist of at least the following elements and is not limited to them: an application programming interface (API) for the application, in particular the mobile device application; data management unit; secure private customer payment info data storage; sequencing settings data storage for public tracks, playlists and albums; high bandwidth interface to manage quick service accessibility.

The server-side database may have an own dedicated hardware with multiple backups of all the stored data with continuously performed data backups.

The communication interface may comprise at least a transmitting unit and a receiving unit to retrieve, collect and exchange data with the mobile device and/or the central server.

In some designs, the communication interface may be configured to provide communication to a back-end device, for example providing a server side database.

In some designs, the light sequencing algorithm may be configured to generate light sequences based on received audio data and/or synchronous to music files and/or audio files playing on the mobile device and/or the central server and/or sensor measurement data and/or user input data.

In some designs, the light sequencing algorithm may be configured to generate light sequences based on received audio data and/or user settings.

In some designs, the light sequencing algorithm may be configured to analyze audio data packages that are received via radio waves, for example on low energy and/or via BLE connection, on the fly, meaning as soon as they arrive. Information generated based on the analyzed audio data packages alongside the input of a number of user input variables from the sequencing settings may then be used to generate light sequences synchronous to the played music file and/or audio file.

In summary: The control system architecture, in particular the software system architecture, may consist of three parts. The first part is a code running on the chip of the operating device of the portable light apparatus, responsible for generating light sequences and communicating via radio waves, in particular using low energy standard protocols and/or via BLE connection with a smartphone or a computer. The smartphone or computer application, as the central device, is the second part responsible for user account verification and data transmission to the glasses and acting as the main user interface/input device for the portable light apparatus. The third part of the system is a server back-end storing user data like payment info to verify and activate the use of the portable light apparatus as well as user account settings and data related to the use of the portable light apparatus. Because the desired stroboscopic light effect is best combined with auditory stimulation of the user, the light sequences will be automatically generated “on the fly” based on audio data input via low energy audio data and user preferred settings that may be controlled using the smartphone or computer application. Thus, no information is required for the light sequences to be encoded into a music file and/or an audio file, for example on different radio waves. In some designs, a brightness of the LEDs may be able to be manipulated through the normal user inputs used to regulate volume of a loudspeaker or headphones. Using the low energy audio standard, for example the BLE connection, the same audio data may be transmitted wirelessly to an actual loudspeaker or pair of headphones and the glasses at the same time. To be able to receive any incoming audio data, sent from the central device's operating system (OS) to the portable light apparatus, the portable light apparatus has to advertise themselves to the central device as speakers or headphones. The user settings for automated light sequence generation may be the number and order of effects with a frequency band for each effect and transitions between the effects or music titles and/or audio titles. The effects and transitions may be chosen from an effect library and manipulated to fit the user's wishes and needs—for example the stroboscopic frequency of the effect. Therefore, the light sequencing algorithm may generate stroboscopic light sequences and/or frequencies on the fly based on audio data packages.

In some designs, a storage device may be provided to store user input data and/or music files and/or audio files.

According to at least one of the above described embodiments, the electronic device and the light device are configured to emit light with adjustable light color. For example, the light emitting diodes are configured to emit light with a wavelength greater than 380 nm and smaller than 800 nm/or chosen from the group consisting of red, green, blue, yellow, orange, violet. In some configurations the light emitting diodes may have at least two adjustable channels for specific light colors, such as additional red light with wave lengths in the band of 600 nm to 800 nm. In other configurations so called RGB (red, green, blue) LEDs may be configured as part of the light device and in some configurations even RGBW (red, green, blue, white) light LEDs may be used. The light emitting diodes are configured to not emit light in the ultraviolet or infrared wavelength band to be compliant with eye safety regulation standards such as IEC 62471 and/or ANSI/IESNA RP-27. To adjust the emitted light color and mix of wavelengths and wavelength bands, the electronic device may receive direct input from an external control device, such as a computer, a laptop and/or a smartphone, the input may then be used to control the light device with its configured LEDs.

The invention further relates to a control system of the above described portable light apparatus and a mobile device, e.g. a smartphone or tablet, wherein the portable light apparatus comprises a communication unit, the above described light device and the above described electronic device and the mobile device comprises a communication unit and a light therapy application. Preferably, the light therapy application is configured to perform a communication connection, e.g. a wireless communication path, e.g. a Bluetooth connection, via the communication units to the electronic device of the portable light apparatus from the mobile device to control at least one of the following light parameters brightness, strobe frequency and light color of the light device. The light therapy application is a software which is an application/app installed on the mobile device. The light therapy application is configured to wirelessly communicate with the operating device and to control the light device, e.g. each of the LEDs. For example, the light therapy application may be configured to raising or lowering the light brightness, to change light color and/or to change strobe frequency, e.g. based on a predetermined ambient light setting. The light therapy application may be configured to track the general use and use of the different modes of operation applied by the user providing insight and overview as well as to keep track of progress with the therapy. The light therapy application may also provide features for creating and storing custom light settings and sequences such as specific brightness, color and strobe frequency settings.

The light therapy application may also provide features for information exchange between users, and user groups such as light sequence creators, recreational users, patients and doctors. The light therapy application may also provide features to control access to different functions of the portable light apparatus by using a so called “log-in” system.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings which are given by way of illustration only, and thus, are not limiting the present invention, and wherein:

FIG. 1 schematically illustrates a perspective view of an embodiment of a portable light apparatus, comprising a front frame, a pair of side frames and an operating device having at least a battery and at least one electronic part having a light device, wherein the battery is arranged in one of the side frames and the electronic part and the light device is arranged in the front frame,

FIG. 2 schematically illustrates a perspective view of an embodiment of a portable light apparatus, comprising a front frame, a pair of side frames and an operating device having a battery, a main electronic part and an auxiliary electronic part with a light device, wherein the battery and the main electronic part are arranged in one of the side frames and the auxiliary electronic part and the light device are arranged in the front frame,

FIG. 3 schematically illustrates a detailed perspective view of the portable light apparatus according to FIG. 2,

FIG. 4 schematically illustrates a perspective side view of the portable light apparatus according to FIG. 2,

FIG. 5 schematically illustrates a perspective view of an embodiment of a charging case for a portable light apparatus, the charging case comprising at least a housing and a cavity in which the portable light apparatus is insertable to be held, protected and charged,

FIG. 6 schematically illustrates a front view of an embodiment of an electronic part with a light device, comprising four light emitting diodes which emission cones intersect in an intersection area for indirect illumination,

FIG. 7 schematically illustrates a side view of the emission cones and their intersection area according to FIG. 6,

FIG. 8 schematically illustrates a representation of a plane of a spectacle front and planes of eyeglass shapes of a frame (view from above),

FIG. 9 schematically illustrates a perspective view of an enlarged section of the auxiliary electronic part according to FIG. 6,

FIG. 10 schematically illustrates another perspective view of an enlarged section of the auxiliary electronic part according to FIG. 6,

FIG. 11 schematically illustrates a control system of a portable light apparatus and a mobile device to control the light device,

FIG. 12 schematically an embodiment of a control and operating architecture,

FIG. 13 schematically illustrates an exploded view of a portable light apparatus, comprising a front frame, a pair of side frames and an operating device having a battery and an electronic part with a light device,

FIG. 14 schematically illustrates in cross section a top view of an embodiment of a portable light apparatus, comprising a front frame, a pair of side frames and an operating device having a battery and an electronic part with a light device and

FIG. 15 schematically illustrates in cross section a perspective view of the portable light apparatus according to FIG. 14.

Corresponding parts are marked with the same reference symbols in all figures.

### DETAILED DESCRIPTION OF SPECIFIC EMBODIMENTS

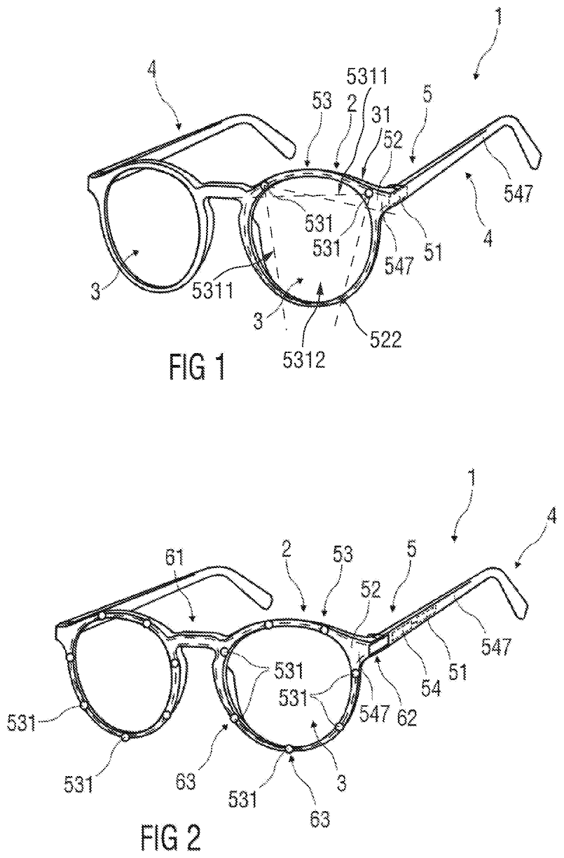

FIG. 1 shows schematically a perspective view of an embodiment of a portable light apparatus 1, in particular glasses, configured to be worn by a user.

The portable light apparatus 1 comprises a front frame 2 to house glasses 3 and a pair of side frames 4 connected to the front frame 2. The portable light apparatus 1 further comprises an operating device 5 having a battery 51 and an electronic part 52, such as an auxiliary electronic part 52, having a light device 53 configured to emit light towards the glasses 3 and/or the user's eyes. The light device 53 may comprise a number of light emitting diodes 531. The light emitting diodes 531, also called LEDs 531 may be arranged distributed on, in and/or over the front frame 2. The LEDs 531 may be are arranged on a plain surface of the electronic part 52.

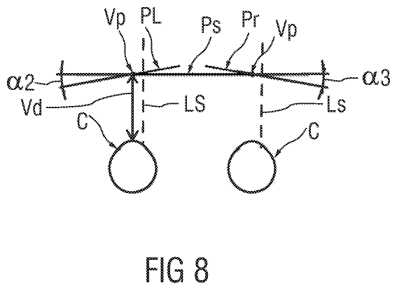

In the shown embodiment, the light device 53 comprises two light emitting diodes 531 assigned to each glass 3, wherein only one pair of light emitting diodes 531 is shown. The light emitting diodes 531 are each arranged in one corner area of an opening 522 of the electronic part 52. The opening 522 aligns with an opening or window of the front frame 2. The light emitting diodes 531 are arranged relative to one another such that their light emission cones 5311 substantially intersect between a back surface 31 of the respective glass 3 and an apex of the user's cornea C (corneal apex), schematically shown in FIG. 8, to indirectly illuminate a line of sight Ls of the user, as shown in FIG. 8. The corneal apex is a point of maximum curvature on the cornea. The line of sight Ls is a visual axis or sightline between the user's eye(s) and a subject.

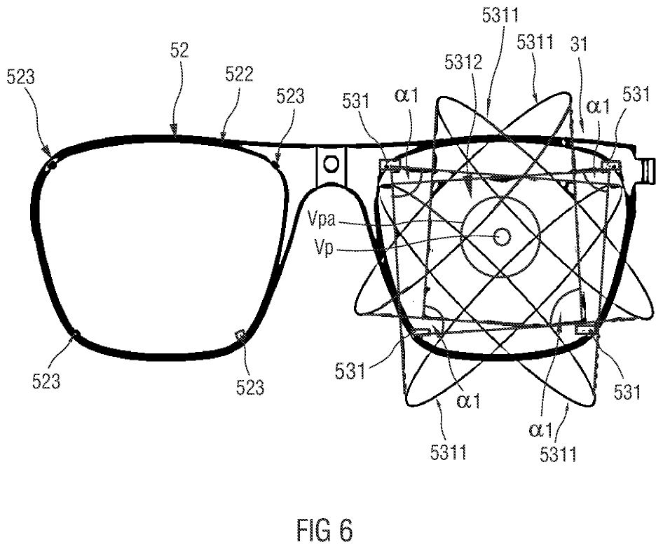

The light emitting diodes 531 may be so called Chip LEDs. The emission cones 5311 each may have an opening angle α1 (shown in FIG. 6), beam angle or radiation angle of less than or equal to 90°. The light emitting diodes 531 emits light in a shape of a cone.

The emission cones 5311 intersect in an intersection area 5312 which covers a large area in relation to the respective glass 3 and a large area about and along a vertex distance Vd, as shown in FIG. 8.

In some designs, the LEDs 531 may be arranged partially or completely surrounding and/or encasing the front frame recesses for the glasses 3. In some designs, the light device 53 is arranged in rims forming the front frame 2 for holding the glasses 3.

The battery 51 is configured to supply energy to the light device 53 via the electronic part 52. The battery 51, in particular a rechargeable battery 51, is arranged in one of the side frames 4 and the electronic part 52 and the light device 53 are arranged in the front frame 2.

The electronic part 52 may fully have the shape of the front frame 2. The electronic part 52 may comprise LEDs 531 equally distributed around both eyeglasses 4 of the portable light apparatus 1.

Alternatively, the electronic part 52 may have a shape of half of the front frame 2, for example in an eyeglass holding region. In some designs, two electronic parts 52 may be arranged in the front frame 2. Each electronic part 52 may have the shape of one eyeglass holding region. Each of the electronic part 52 may comprise a respective light device 53.

Moreover, optionally additionally the portable light apparatus 1 may comprise at least one audio output device 547. The audio output device 547 is configured to output audio to the user.

The audio output device 547 may be arranged in at least one of the side frames 4. The audio output device 547 may be arranged in an area of the side frame 4 which is close to a user's ear when worn by the user. The audio output device 547 may comprise at least one loudspeaker and/or earphone and/or a bone sound transducer.

The audio output device 547 may at least be partially arranged in the front frame 2. In some designs, at least one loudspeaker, earphone and/or bone sound transducer may be arranged in the front frame 2. The audio output device 547 may be connected to an electronic part 54, such as a main electronic part 54. The audio output device 547 may comprise at least one interface to communicate with an external control device.

FIG. 2 shows schematically a perspective view of an embodiment of a portable light apparatus 1, comprising a front frame 2, a pair of side frames 4 and an operating device 5 having a battery 51, a main electronic part 54 and an auxiliary electronic part 52 with a light device 53, wherein the battery 51 and the main electronic part 54 are arranged in one of the side frames 4 and the auxiliary electronic part 52 and the light device 53 are arranged in the front frame 2.

In some designs, the auxiliary electronic part 52 is a flexible printed circuit board (flexible PCB, FPCB) which has an outline shape of the front frame 2. In some designs, the main electronic part 54 is a rigid printed circuit board (main PCB) with a number of electronic components.

In some designs, the auxiliary electronic part 52 comprises one bent area 61 in a nose bridge area. In some designs, the auxiliary electronic part 52 comprises a bent area 62 in a connection area 521 to at least one of the side frames 4, in particular to the main PCB. In some designs, the auxiliary electronic part 52 comprises a bent area 63 in an area of at least one light emitting diode 531 of the light device 53 to direct emitted light by the light device 53 towards the user's eye.

Moreover, optionally additionally the portable light apparatus 1 may comprise at least one audio output device 547. The audio output device 547 is configured to output audio to the user.

The audio output device 547 may be arranged in at least one of the side frames 4. The audio output device 547 may be arranged in an area of the side frame 4 which is close to a user's ear when worn by the user. The audio output device 547 may comprise at least one loudspeaker and/or earphone and/or a bone sound transducer.

The audio output device 547 may at least be partially arranged in the front frame 2. In some designs, at least one loudspeaker, earphone and/or bone sound transducer may be arranged in the front frame 2. The audio output device 547 may be connected to the main electronic part 54. The audio output device 547 may comprise at least one interface to communicate with an external control device.

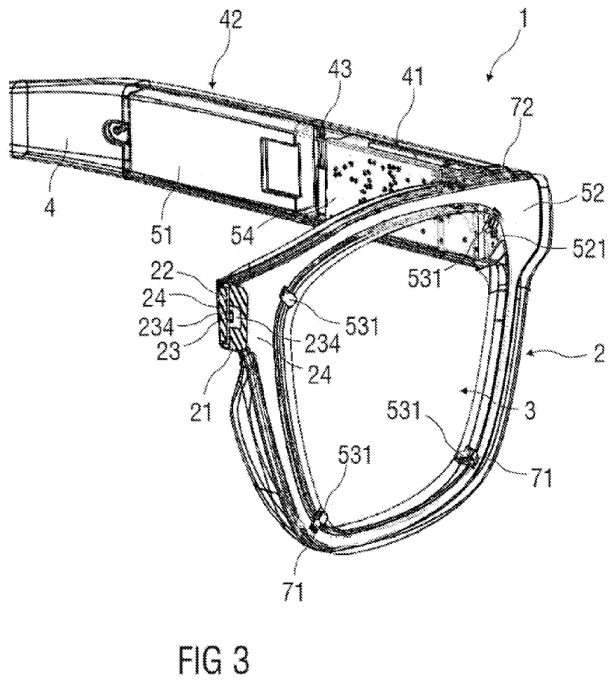

FIG. 3 shows schematically a detailed perspective view of the portable light apparatus 1 according to FIG. 2.

In some designs, the flexible auxiliary electronic part 52 has one stiffener layer 71 located in an area underneath the light device 53, in particular underneath each light emitting diode 531 of the light device 53. Each light emitting diode 531 is soldered to the flexible auxiliary electronic part 52.

In some designs, the flexible auxiliary electronic part 52 has one stiffener layer 72 located in an area underneath, above or both at the bent area 62 of the connection area 521 to at least one of the side frames 4 and/or to the main PCB to support the bend and connection.

The flexible auxiliary electronic part 52 is configured advantageously thin, so it may fit into any front frames 2 of glasses, in particular regular glasses, without changing its design and appearance as regular everyday glasses. The stiffener layers 71, 72 may support the flexible PCB in connection regions with other components of the portable light apparatus 1. The stiffener layers 71, 72 may be joined to the flexible PCB, for example by soldering or glue. The stiffener layers 71, 72 may be coatings or separate support plates.

The front frame 2 comprises two frame parts 21, 22, for example a front frame part 21 and a rear frame part 22, which are permanently or temporarily connected to one another to hold the glasses 3.

In some designs, a fastening device 23 consisting of two corresponding fastening elements 231 is arranged in a nose bridge 24 of the front frame 2. The fastening device 23 may be one of a form-fitting device, a force-fitting device and/or material-fitting device for either a temporary or permanent connection of the frame parts 21, 22. The fastening elements 231 may be clip elements, clamping elements, snap-fit elements, adhesive elements, welding elements, screws, rivets or the like. In the shown embodiment the fastening elements 231 are configured as attracting magnet elements. The fastening device 23 is configured as a safe and reliable connection, in particular magnetic connection for simply detachable and connectable parts.

The frame parts 21, 22 are configured to hold and house the glasses 3 and the auxiliary electronic part 52 with its light device 53. The front frame 2 is configured to house and embed the glasses 3 and the auxiliary electronic part 52.

In one configuration, the front frame part 21, for example in in the area of the nose bridge 24, may be in cross-section substantially U-shaped or the like. The fastening elements 231 may be arranged in a respective recess formed in the nose bridge 24 of the front frame part 21 facing towards the rear frame part 22. The rear frame part 22 may have a respective size and shape corresponding with the form of the front frame part 21 in the area of the nose bridge 24. The nose bridge 24 of the rear frame part 22 is, for example, shaped as a bridge or web.

The front frame parts 21, 22 may comprise corresponding positioning elements, such as positioning pins and holes, clips, screws, rivets or other fastening and/or positioning elements. Alternatively or optionally additionally, the front frame parts 21, 22 may be coupled to each other by adhesive bonding, welding such as ultrasonic welding, soldering, overmolding and/or by a filling material such as potting compound.

The front frame 2 comprises two windows to encase the glasses 3. The flexible auxiliary electronic part 52 may also comprise openings, windows or cutouts corresponding with the size and shape of the windows of the front frame 2 so as to not block a vision of the user. The LEDs 531 of the light device 53 are also arranged in the front frame 2, in particular around the respective window, such that they do not block vision of the user when looking through the glasses 3.

In the respective side frame 4 in which the battery 51 and the main electronic part 54 are arranged and safely housed, the main electronic part 54 is arranged in a front region 41 of the side frame 4 which is closer to the connection area 521 to the front frame 2. The battery 51 is located in a rear region 42 adjacent or behind the main electronic part 54. The main electronic part 54 connects the battery 51 with the flexible auxiliary electronic part 52 for energy control and energy supply. The connection between battery 51 and the main electronic part 54, that means to the main PCB, may achieved by one of a common connection technique, for example via a cable device.

The respective side frame 4 may comprise a recess 43 to hold and encase the battery 51 and the main electronic part 54. The respective side frame 4 may also consist of two side frame parts which are permanently or temporarily coupled to each other. In some designs, the side frame parts may be joined together with magnets, snap fittings, glue, friction welding, overmolding or any other temporarily or permanent joining technique. Alternatively, the side frame 4 is configured as one-piece web or bridge housing respective components of the operating device 5.

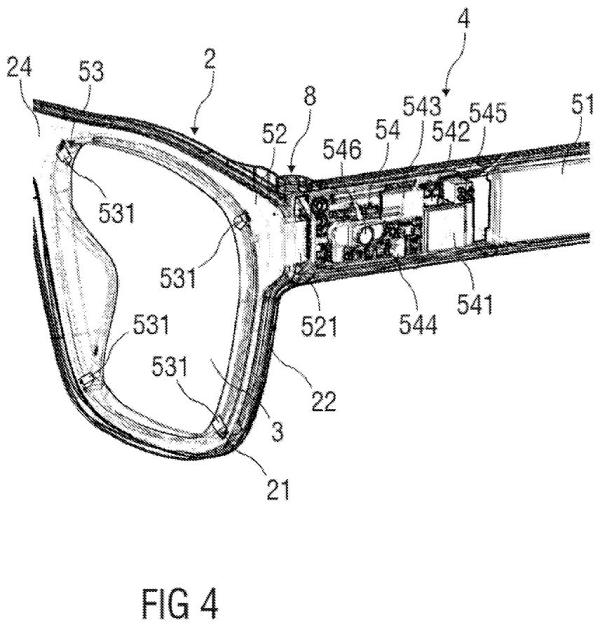

FIG. 4 shows schematically a perspective side view of the portable light apparatus 1 according to FIG. 2.

The front frame 2 is mechanically connected to the side frames 4, for example via a hinge joint 8 or any other connection mechanism. In some designs, the connection mechanism may be configured as a hinge and slide mechanism, for example a rotary-sliding hinge. In some designs, a pivot joint is configured as a sliding joint at the same time, wherein pivot movement of the side frame 4 relative to the front frame 2, when folding from the use state to the non-use state of glasses, causes the side frame 4 to slide relative to the front frame 2 or vice versa. The side frames 4 are foldable in a direction towards and away from the front frame 2. In some designs, one of the frame parts 21, 22 is connected to the side frames 4 via a respective hinge joint 8.

The portable light apparatus 1 may comprise a different wearing element than the side frames 4 connected to the front frame 2 to be worn by a user. The wearing element may be made from textile material. The wearing element may be a rubber band, headband or sweatband or the like to be worn by a user. The wearing element may embed, for example between different textile layers, at least the main electronic part 54 and the battery 51.

In an area of the respective hinge joint 8 between front frame 2 and the side frame 4, the flexible auxiliary electronic part 52 is connected to the main electronic part 54.

In some designs, the flexible auxiliary electronic part 52 is bent to create the connection area 521 between the flexible auxiliary electronic part 52 and the main electronic part 54. The connection area 521, in particular a connection and power interface, is arranged substantially perpendicular to a surface of a rear side facing towards the side frames 4 of the flexible auxiliary electronic part 52.

The operating device 5 comprises a processor module 541 on a chip held on the main PCB. The processor module 541 may comprise one or more processing cores. The operating device 5 comprises a radio module 542 coupled to the one or more processing cores to transmit and exchange data using radio waves. In some designs, the radio module 542 is configured to transmit and exchange data using low energy, for example using a Bluetooth Low Energy (BLE) connection. This is, for example, known from audio streaming over radio waves. In some designs, BLE Audio is configured to stream audio data.

Further, the operating device 5 comprises a light device driver 543 held on the main PCB to control stroboscopic frequency and/or light sequences of the light device 53. The main PCB may hold a multi-channel driver chip as the light device driver 543 to receive logic level signals from the processor module 541 to control stroboscopic frequency, for example for generating a light sequence, of the light device 53.

Further, the operating device 5 comprises a voltage regulation and battery charging circuitry 544 held on the main PCB to power the processor module 541 and the light device 53 on the flexible PCB and to recharge the battery 51.

In some designs, the operating device 5 comprises a charging connector 545, also called charging port or charging interface, which is arranged on the respective side frame 4. In one configuration, the charging connector 545 is arranged substantially perpendicular to a surface of the side frame 4. In another configuration, mating surfaces between the side frame 4 and the front frame 2 may house different charging interface elements. This arrangement allows a simple connection with a recharging device in a folded state of the portable light apparatus 1. The folded state refers to a commonly known fold technique of glasses.

In a further example, the operating device 5 comprises power switch 546 to connect a circuit of the main PCB to the battery 51 and to connect or disconnect all electrical components of the operating device 5 except the charging circuitry from the battery 51.

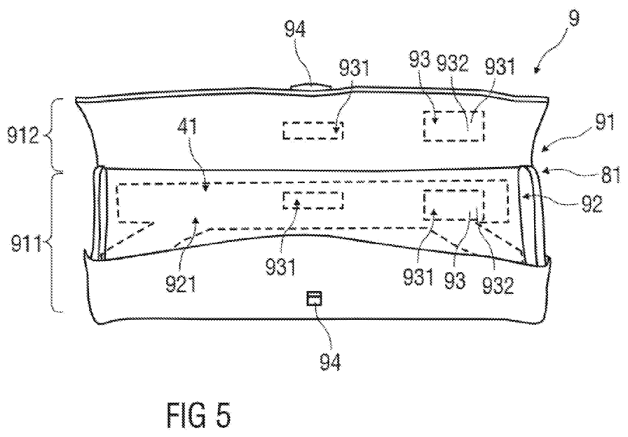

FIG. 5 shows schematically a perspective view of an embodiment of a charging case 9 for a portable light apparatus 1, the charging case 9 comprising a housing 91 and a cavity 92 in which the portable light apparatus 1 is insertable to be held, protected and charged.

The charging case 9 further comprises a charging operating device 93 arranged in the housing 91, wherein the charging operating device 93 is configured to charge the battery 51 of the portable light apparatus 1 when inserted in the cavity 92.

In some designs, the housing 91 comprises two corresponding housing parts 911, 912, for example a base housing part 911 and a secondary housing part 912 such as a flap or lid. The housing 91 and/or the housing parts 911, 912 may be made from hard shell and/or soft shell. One of the housing parts 911, 912, for example the base housing part 911, forms the cavity 92 and the other one of the housing parts 911, 912, for example the secondary housing part 912, is configured to close or open the cavity 92. The housing parts 911, 912 may be connected to each other by a hinge joint 81 to form the cavity 92 that may be opened and closed.

The housing parts 911, 912 contain magnets 94 that are oriented to attract the respective housing parts 911, 912. The housing parts 911, 912 may alternatively or optional additionally comprise other fasteners, such as clips and/or garment fasteners or the like.

In some designs, the cavity 92 is formed such that an insert direction, according to arrow A1, of the portable light apparatus 1 is defined for the user. The cavity 92 may be shaped such that the portable light apparatus 1 is tightly inserted and fitted in the cavity 92. The specified insert direction may refer to a specified folding configuration of the side frames 4. In some designs, the base housing part 911, in particular the cavity 92, may comprise a shaped recess 921 defining side frames 4 in a folded state.

In some designs, at least one of the housing parts 911, 912 comprises a holding element 913 configured to guide the user to insert and place the portable light apparatus 1 and to removably hold the portable light apparatus 1 in place in the cavity 92. In some designs, the holding element 913 is a magnet element which is arranged in an area of one of the housing parts 911, 912 corresponding with the location of a counter holding element located on the front frame 2 and/or the side frames 4 of the portable light apparatus 1. In some designs, the counter holding element may be provided by the integrated fastening device 23, for example the fastening elements 231, of the front frame 2.

The charging operating device 93 comprises a charging port 931 or connector which may be arranged in at least one of the housing parts 911, 912 to establish a temporary electrical connection to the portable light apparatus 1. The charging operating device 93 comprises charging electronics 932 which may be arranged in at least one of the housing parts 911, 912 for charging the battery 51 of the portable light apparatus 1. Charging electronics 932 may comprise at least one battery, such as a rechargeable battery.

FIG. 6 shows schematically a front view of an embodiment of an auxiliary electronic part 52 with a light device 53, comprising four light emitting diodes 531 which emission cones 5311 intersect in an intersection area 5312 for indirect illumination.

The light emitting diodes 531, also called LEDs 531 are arranged distributed on, in and/or over the auxiliary electronic part 52, in particular with respect to each opening 522.

In the shown embodiment, the light device 53 comprises four light emitting diodes 531 assigned to each glass 3. The light emitting diodes 531 are each arranged in one corner area of the opening 522 of the auxiliary electronic part 52. The opening 522 aligns with an opening or window of the front frame 2. The light emitting diodes 531 are arranged relative to one another such that their light emission cones 5311 substantially intersect between a back surface 31 of the respective glass 3 and an apex of the user's cornea C (corneal apex), schematically shown in FIG. 8, to indirectly illuminate a line of sight Ls of the user, as shown in FIG. 8. The corneal apex is a point of maximum curvature on the cornea. The line of sight Ls is a visual axis or sightline between the user's eye(s) and a subject.

The light emitting diodes 531 may be so called Chip LEDs. In some designs the emission cones 5311 each may have an opening angle α1 of less than or equal to 90°, in some designs the angle α1 may be equal to or greater than 90°. The light emitting diodes 531 emits light in a shape of a cone.

The emission cones 5311 intersect in an intersection area 5312 which covers a large area in relation to the respective glass 3 and a large area about and along a vertex distance Vd, as shown in FIG. 8. Moreover, the intersection area 5312 covers a large area of a visual point Vp and a visual point area Vpa.

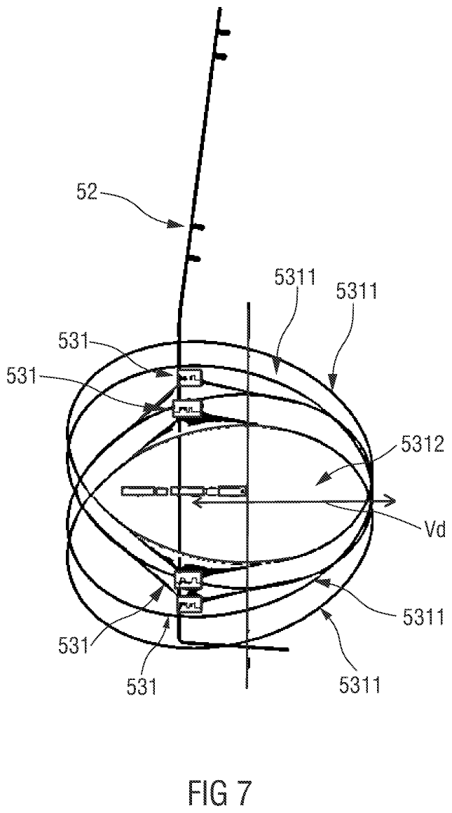

The light emitting diodes 531 are arranged angled relative to one another such that the intersection area 5312 of the emission cones 5311 is substantially elliptical shaped, as shown in FIG. 7.

The light emitting diodes 531 are arranged aligned relative to one another such that the intersection area 5312 of their emission cones 5311 covers a length range between 5 mm and 35 mm between the back surface 31 of the respective glass 3 and the apex of the user's cornea C, as shown in FIG. 8.

The auxiliary electronic part 52 comprises two openings 522 to receive the glasses 3 and a number of projecting tabs 523 arranged distributed in an area of the openings 522. Each tab 523 is connected to one of the light emitting diodes 531. In other words: each light emitting diode 531 is attached to one of the tabs 523.

The tabs 523, such as flaps or lugs, are located in corner areas of each opening 522. Depending on a desired arrangement and/or orientation of the LEDs 531 and their emission cones 5311, the tabs 523 may vary in their numbers and/or arrangements.

FIG. 7 shows schematically a side view of the emission cones 5311 and their intersection area 5312 according to FIG. 6. The emission cones 5311 are oriented with respect to each other such that the intersection area 5312 is substantially elliptical shaped to cover a large area of the vertex distance Vd.

FIG. 8 shows schematically a representation of a plane Ps of a spectacle front and planes Pr, Pl of eyeglass shapes of a front frame 2 (view from above). The visual points Vp refer to points of intersection of vertical centerlines of the plane Pl of a left glass 3 and the plane Pr of the right glass 3 with the plane Ps of the spectacle front. The planes Pr, Pl of the right glass 3 and the left glass 3 may be angled by a predefined angle α2, α3 with respect to the plane Ps of the spectacle front, for example of the front frame 2. The vertex distance Vd distance is a distance between the back surface 31 of the glass 3 and the apex of the cornea C, measured with the line of sight Ls perpendicular to the plane Ps of the spectacle front.

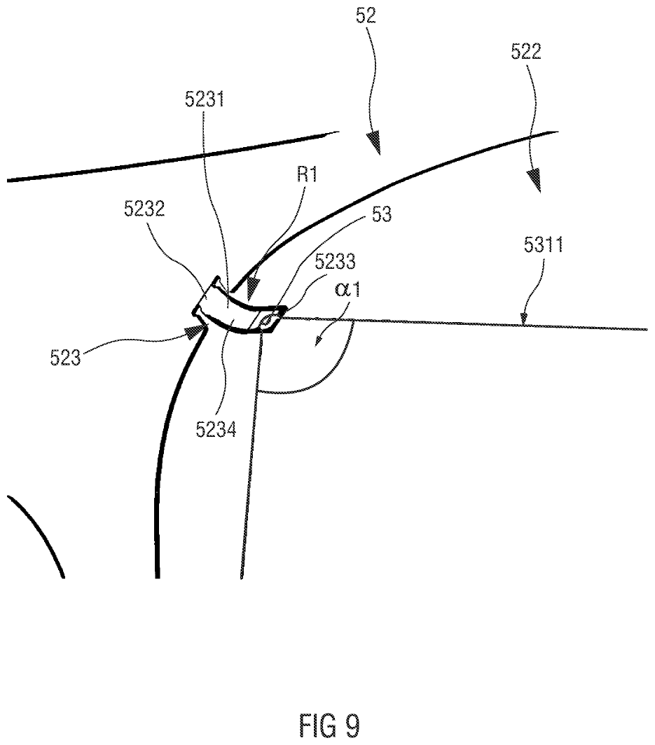

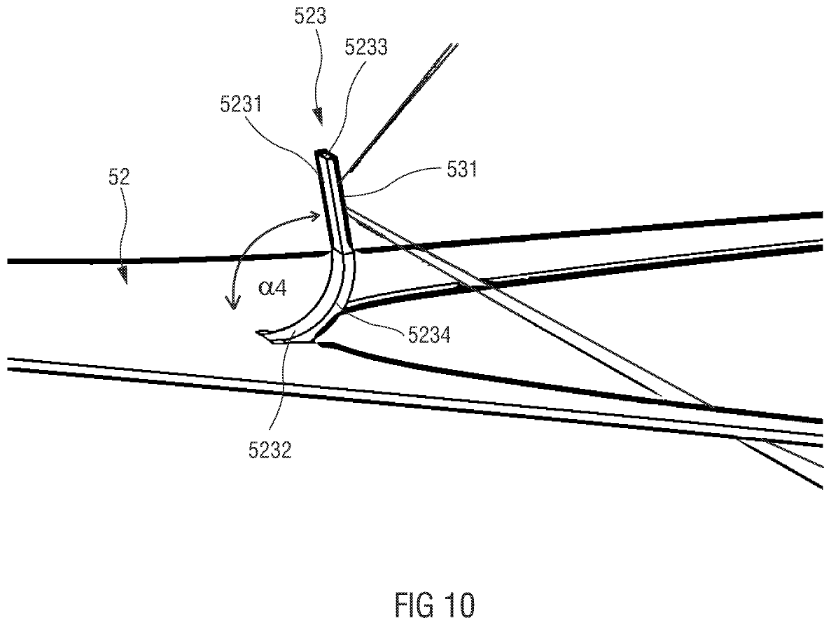

FIG. 9 shows schematically a perspective view of an enlarged section of the auxiliary electronic part 52 and FIG. 10 shows schematically another perspective view of an enlarged section of the auxiliary electronic part 52.

Each tab 523 comprises a main tab body 5231 having a first end 5232 connected to the opening 522, i.e. to the auxiliary electronic part 52, in particular to the flexible circuit board, and a second free end 5233 extending away from the opening 522. A respective light emitting diode 531 is attached or mounted to the second free end 5233. The tabs 523 may be formed out, punched out, cut out or molded from the auxiliary electronic part 52, in particular from the flexible circuit board, and bent with respect to a plane of the auxiliary electronic part 52.

Each tab 523 has a bent portion 5234 arranged between the first end 5232 and the second free end 5233. Each tab 523 is bent about a bending radius R1 (shown in FIG. 9), for example smaller than 1 mm, in particular of 0.5 mm.

Each tab 523 is bent projecting away from a front surface (directed away from a face of the user) and towards a back surface (directed towards a face of the user) of the auxiliary electronic part 52 about an acute angle α4 (shown in FIG. 10). The acute angle α4 may be between 45° to 89°, for example 60° to 85°, in particular 80°.

It is to be understood that the bending radius R1 and/or the angle α4 of each of the tabs 523 may vary between each of the tabs 523 and/or depending on a desired orientation of the emission cones 5311 and/or desired illumination of an intersection area 5312.

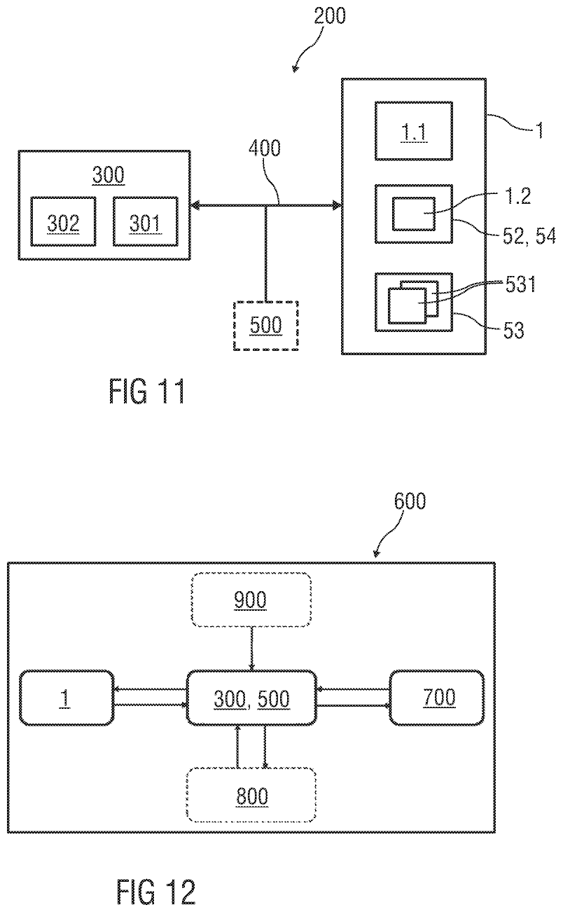

FIG. 11 shows schematically illustrates a control system 200 comprising at least the portable light apparatus 1 and a mobile device 300 to control the light device 53.

An electronic device, e.g. the auxiliary electronic part 52 and/or the main electronic part 54, and the light device 53 are configured to emit light with adjustable light color. For example, the light emitting diodes 531 are configured to emit light with a wavelength band greater than 200 nm, e.g. 300 nm to 800 nm, and/or chosen from the group consisting of red, green, blue, yellow, orange, violet.

The portable light apparatus 1 comprises a communication unit 1.1, e.g. a Bluetooth interface, and a light therapy operation mode 1.2 implemented in one of the electronic parts 52, 54 as described above.

The mobile device 300 comprises a respective communication unit 301 and a light therapy application 301 implemented a software module or a so-called App.

Preferably, the light therapy application 302 of the mobile device 300 is configured to perform a communication connection 400, e.g. a wireless communication path, e.g. a Bluetooth connection, via the communication units 1.1, 301 to the electronic parts 52, 54 of the portable light apparatus 1 from the mobile device 300 to control at least one of the following light parameters brightness, strobe frequency and light color of the light device.

The light therapy application 302 is a software which is an application/app installed on the mobile device 300. The light therapy application 302 is configured to wirelessly communicate with the operating device 5 and to control the light device 53, e.g. each of the LEDs 531. For example, the light therapy application 302 may be configured to raising or lowering the light brightness, to change light color and/or to change strobe frequency, e.g. based on a predetermined ambient light setting.

The control system 200 provides a remote control for the portable light apparatus 1 to be worn by the user, the control system 200 comprises at least a light sequencing algorithm implemented as the light therapy application 302 on the mobile device 300 and/or as light therapy operation modes on one of the electronic parts 52, 54 of the portable light apparatus and a communication interface based on a communication connection 400.

The communication connection 400 provides a connection path between the mobile device 300 the portable light apparatus 1 and optionally to a central server 500.

Depending on data transmitted from the mobile device 300 and/or from the central server 400 directly or indirectly via the communication unit 1.1 to the operating device 5 of the portable light apparatus 1 a light sequencing algorithm of one of the light therapy operation modes 1.2 generates a light sequence for the light device 53, e.g. the LEDs 531.

The control system 200 may enable remote control for the portable light apparatus 1 so that the portable light apparatus 1 may be controlled from remote mobile devices 300 to allow a simple user interface. Furthermore, the control system 200 is configured to enable user engagement with the portable light apparatus 1 and social engagement between individual customers for example on a centralized platform for their own portable light apparatus 1.

The control system 200 may be configured as a processor module integrated as the light therapy application 302 in the mobile device 3, as a control application in the central server 500 and/or as a control application in the processor module 541 of the portable light apparatus 1, for example of the operating device 5, to run the portable light apparatus 1. The control system 200 is an operating system and may be configured as software.

The control system 200 is configured to be responsible to process at least one of the following described tasks. The control system 200 may be configured to receive audio data from an external source, such as the mobile device 300 and/or the central server 500, via radio waves and/or optical waves. The control system 200 may be configured to manage software updates, wireless or wired. The control system 200 may be configured to retrieve, collect and exchange user data with the external source based on user settings. The control system 200 may be configured to store user input variables necessary to generate light sequences of the light device 53 of the portable light apparatus 1. The control system 200 may comprise a user authorization and activation based on input data from the mobile device 300, such as a smartphone or tablet, and/or the central server 500, for example from an application (short: APP) or a server-side device and/or back-end server and/or device, shown in FIG. 11.

The control system 200 may communicate with the mobile device 300 and/or the central server 500 to analyze, for example, played audio files and/or music files on the mobile device 300 and/or the central server 500 and to synchronize and/or generate an appropriate light frequency and/or light sequence of the light device 53 according to the audio file and/or music file listened by the user. The communication connection 400 is configured to exchange audio data based on low energy, for example via radio waves.

The communication connection 400 may be configured to exchange audio data over Bluetooth Low Energy (BLE) connection.

In some designs, the light sequencing algorithm 201 is configured to generate light sequences based on received audio data and/or user settings.

In some designs, the light sequencing algorithm of the light therapy operation mode 1.2 and/or of the light therapy application 302 are/is configured to analyze audio data packages that are received via radio waves on the fly, meaning as soon as they arrive.

Information generated based on the analyzed audio data packages alongside the input of a number of user input variables from sequencing settings may then be used to generate light sequences to the played music file and/or audio file on the mobile device 300 and/or central server 500.

FIG. 12 shows schematically an embodiment of a control and operating architecture 600, also called “Strobes System” for a portable light apparatus 1.

The architecture 600 of a system to control and operate the portable light apparatus 1 comprises multiple main components. The system's hardware consists of the portable light apparatus 1 and the charging case 9. Software may consist of, for example, three subsystems. One subsystem may be embedded software in the portable light apparatus 1. The embedded software may comprise the control system 200 of the portable light apparatus 1. One subsystem may be the mobile device 300 and/or the central server 00. One subsystem may be services provided by the server-side 700.

The whole system may be used in the following way, for example from a perspective of a company: The hardware consisting of the portable light apparatus 1 and the charging case 9 may be shipped to a customer who has created a user account and paid their fees. As hardware the user may additionally use output devices 800, such as headphones or loudspeakers.

In some designs, an automated sequence generator may provide a service for the use of a stroboscopic light sequences. The interaction between the portable light apparatus 1 and the central server 500 may be based on a monthly or an annual subscription payment. The user payment and/or subscription information may be stored in the server-side 700 and will be needed for activation of the stroboscopic light features of the portable light apparatus 1 via the central server 500, which means via an APP.

If the user does not activate the portable light apparatus 1 or continue to pay the subscription after ending of a paid period, all features will be disabled until the payment is continued. Under these circumstances, the portable light apparatus 1 could only be used as regular glasses or sunglasses. The application may provide functionality to collect data anonymously like use duration and use mode, which may enable better understanding of needs of customers.

The application will also have a “voluntary survey for research”—feature to collect more detailed data turning users into probands, which will be the scientific foundation to prove efficacy of “use protocols” for different conditions in clinical trials. The protocols will later be provided in professional license training units for medical professionals, to show how to use the portable light apparatus 1, in particular as light therapy apparatus, for different patient conditions.

Every user will have the “Album” or “Playlist” feature that enables them to store and share the settings they are using with different audio tracks and/or music tracks. The audio tracks and/or music tracks may be stored on the user's mobile device 300 and/or the central server 500. When the mobile device 300 and/or the central server 500 is connected to the output device 800, the user may listen to the respective tracks in combination with the application of light sequences.

The playlists may be made publicly available, be kept in privacy or shared with everyone and/or only selected users. This way, users may share their experience with each other and licensed practitioners will have the ability to share their sequences with their patients.

The album function will also come with a “commentary” and “like” function, for all shared albums. This way all types of users will be able to give each other feedback on their experiences which will help to improve everyone's experiences and enhance the social engagement aspect.

Private album information may be stored locally on the smartphone or computer or be stored in the server-side database to be available and used from devices capable of using audio data, for example on low energy and/or via BLE.

Lastly, a use-case/feature is provided called “party mode” which will be available for all users. This mode may be used locally when multiple people using the so called “Strobes System”, so all portable light apparatus 1 may be synchronized to receive data from a single source so they will generate the same stroboscopic effects for every user. To locally increase signal range and cover large festival areas, long range antennas for the portable light apparatus 1 to receive input data over long distances, for example up to at least 1 km, may be used and integrated. In some designs the so called “Strobes System” may be extended by using a local streaming terminal or router, that houses long range antennas to transmit signals over low energy radio waves. This way, it may not be necessary to include long range antennas into the portable light apparatus 1 decreasing weight, size and potentially saving battery charge therefore enhancing user experience. The same synchronous effect may also be achieved, through streaming data provided by the central server 500 and/or a streaming service 900 to the portable light apparatus 1. This in return may enable large virtual parties around the globe to experience custom sight sequences adapted to their music.

The use of the portable light apparatus 1 is not limited to the above mentioned so called “Strobes System”.

The so called “Strobes System” may be extended by utilizing multiple sensors, for data collection and data input, as well as multiple output devices. The sensors may include but are not limited to: heartrate sensor, blood pressure sensor, blood oxygen sensor, EEG—electroencephalography measurement data and fMRI—functional magnetic resonance imaging. The output devices that may be utilized to extend the “Strobes System” may include, but are not limited to: transcutaneous electrical nerve stimulation devices, electric actuators that create mechanical oscillations which may be felt by the user via touch.

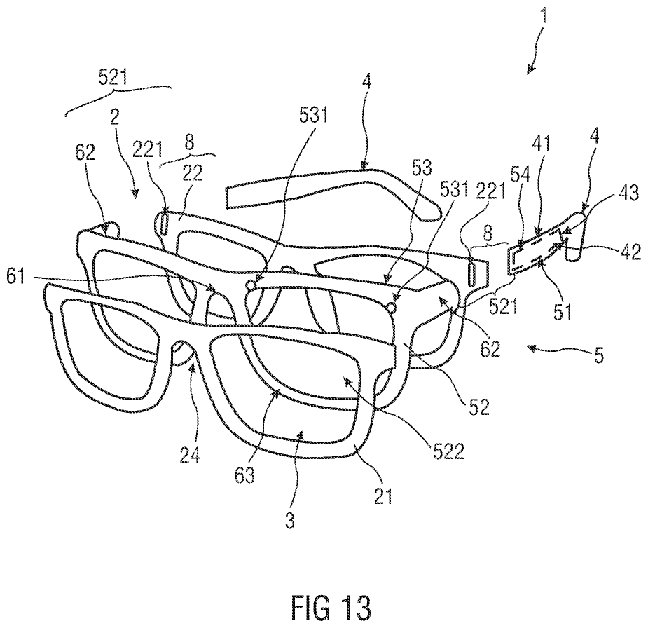

FIG. 13 shows schematically an exploded view of a portable light apparatus 1, comprising a front frame 2, a pair of side frames 4 and an operating device 5 having a battery 51 and an electronic part 52 carrying a light device 53.

The front frame 2 is made of at least two frame parts 21, 22, for example a front frame part 21 and a rear frame part 22, which are permanently or temporarily connected to one another to hold the glasses 3 and the operating device 5, in particular the electronic part 52 and the light device 53. The frame parts 21, 22 are configured to hold and house the glasses 3 and the electronic part 52 with its light device 53. The front frame 2 is configured to house and embed the glasses 3 and auxiliary electronic part 52.

In some designs, the front frame part 21, for example in the area of the nose bridge 24, may be in cross-section substantially U-shaped or the like. The fastening elements 231 may be arranged in a respective recess formed in the nose bridge 24 of the front frame part 21 facing towards the rear frame part 22. The rear frame part 22 may have a respective size and shape corresponding with the form of the front frame part 21 in the area of the nose bridge 24. The nose bridge 24 of the rear frame part 22 is, for example, shaped as a bridge or web.

The front frame parts 21, 22 may comprise corresponding positioning elements, such as positioning pins and holes, clips, screws, rivets or other fastening and/or positioning elements. Alternatively or optionally additionally, the front frame parts 21, 22 may be coupled to each other by adhesive bonding, welding such as ultrasonic welding, soldering, overmolding and/or by a filling material such as potting compound. In some designs, the electronic part 52 may be overmolded and/or back-molded by at least one substrate forming one of the frame parts 21, 22. Afterwards, the other one of the frame parts 21, 22 can be attached, e.g. by clips or screws or the like, to the molded frame part 21, 22. Alternatively, the other one of the frame parts 21, 22 may be formed by overmolding and/or front-molding onto the electronic part 52 and the molded frame part 21, 22.

Due to the thin and flexible configuration of the electronic part with the light device, the light apparatus may maintain its optical appearance as common, wearable eyeglasses compared to other light therapy devices.

The front frame 2 comprises two windows to encase the glasses 3. The electronic part 52, in particular the flexible electronic part 52, may also comprise openings, windows or cutouts corresponding with the size and shape of the windows of the front frame 2 so as to not block a vision of the user. The LEDs 531 of the light device 53 are arranged in the front frame 2, in particular distributed around the respective window and glasses 3, such that they do not block vision of the user when looking through the glasses 3.

In the respective side frame 4 in which the battery 51 and the main electronic part 54 are arranged and safely housed, the main electronic part 54 is arranged in a front region 41 of the side frame 4 which is closer to the front frame 2. The battery 51 may be located in a rear region 42 adjacent or behind the main electronic part 54. It is to be understood that the battery 51 and/or the main electronic part 54 each or both may be arranged within a space provided anywhere at least in one of the front frame 2 or the side frames 4. The main electronic part 54 connects the battery 51 with the flexible electronic part 52, e.g. an auxiliary electronic part 52, for energy control and energy supply. The connection between battery 51 and the main electronic part 54, that means to the main PCB, may achieved by one of a common connection technique, for example via a cable device.

The respective side frame 4 may comprise a recess 43 to hold and encase the battery 51 and the main electronic part 54. The respective side frame 4 may also consist of two side frame parts which are permanently or temporarily coupled to each other. In some designs, the side frame parts may be joined together with magnets, snap fittings, glue, friction welding, overmolding or any other temporarily or permanent joining technique. Alternatively, the side frame 4 is configured as one-piece web or bridge housing respective components of the operating device 5. The main electronic part 54 may be a PCB (printed circuit board). The main electronic part 54 and the battery 51 may be overmolded and/or back-molded by a substrate forming at least one of the side frame parts. Afterwards, the other one of the side frame parts can be attached, e.g. by clips or screws or the like, to the molded side frame part. Alternatively, the other on of the side frame parts may be formed by overmolding and/or front-molding onto the main electronic part 54 and the battery 51 and the molded side frame part. Due to the thin configuration of the main electronic part 54 and the battery 51, the light apparatus 1 may maintain thin side frames 4, also called temples, and thus its optical appearance as common, wearable eyeglasses compared to other light therapy devices.

The front frame 2 is mechanically connected to the side frames 4, for example via a hinge joint 8 or any other connection mechanism. In some designs, the connection mechanism may be configured as a hinge and slide mechanism, for example a rotary-sliding hinge. In some designs, a pivot joint is configured as a sliding joint at the same time, wherein pivot movement of the side frame 4 relative to the front frame 2, when folding from the use state to the non-use state of glasses, causes the side frame 4 to slide relative to the front frame 2 or vice versa. The side frames 4 are foldable in a direction towards and away from the front frame 2. In some designs, one of the frame parts 21, 22 is connected to the side frames 4 via a respective hinge joint 8.

The portable light apparatus 1 may comprise a different wearing element than the side frames 4 connected to the front frame 2 to be worn by a user. The wearing element may be made from textile material. The wearing element may be a rubber band, headband or sweatband or the like to be worn by a user. The wearing element may embed, for example between different textile layers, at least the main electronic part 54 and the battery 51.

In an area of the respective hinge joint 8 between front frame 2 and the side frame 4, the flexible electronic part 52 is connected to the main electronic part 54.

The flexible electronic part 52 comprises at least one bendable and/or bent, e.g. pre-bent, outer area, here the bent area 62. The electronic part 52 carrying the light device 53 may be a flexible printed circuit or flex circuit, consisting of at least one metallic layer of traces, such as copper, e.g. bonded to a dielectric layer. The bent area 62 may be configured as an extension or extended flap of the electronic part 52. The bent areas 61 to 63 and the electronic part 52 may be made from one-piece. The at least one bent area 62 is configured flexible to follow bending motions of the respective side frame 4, comprising at least one of the main electronic part 54 or the battery 51, relative to the front frame 2.

The electronic part 52 comprises one bent area 61 according to a shape of a nose bridge 24. The electronic part 52 comprises at least one bent area 62 forming a connection area 521 to at least one of the main electronic part 54 and/or the battery 51 arranged in one of the side frames 4. The electronic part 52 may be connected to the electronic part 54 and/or the battery 51 via the connection area 521 arranged on one end of the bent area 62. The electronic part 52 comprises a bent area 63 to shape one opening 522 for each of the glasses 3.

In some designs, both side frames 4 may comprise a main electronic part 54 and/or a battery 51 each. Therefore, to provide connection between the flexible electronic part 52 and the main electronic parts 54 and/or batteries 51, both sides of the flexible electronic part 52 may comprise a bent area 61, in particular pre-bent area and reversible bendable area, each. In some designs, only one of the side frames 4 is equipped with a main electronic part 54 and/or a battery 51. Thus, only one side, e.g. edge, of the flexible electronic part 52 may comprise a bent area 62.

The bent area 62 is configured to create the connection area 521 between the flexible auxiliary electronic part 52 and the main electronic part 54. The bent area 62 may be configured in shape of a flat ribbon cable. The connection area 521, in particular a connection and power interface, is arranged substantially perpendicular to a surface of a rear side facing towards the side frames 4 of the flexible electronic part 52. The bent area 62 of the electronic part 52 may be arranged extending into an inside, in particular into the recess 43, of the respective side frame 4 housing the main electronic part 54 and/or the battery 51.

The rear frame part 22 of the front frame 2 may comprise at least one laterally arranged through-hole 221. The electronic part 52 may comprise at least one laterally arranged bent area 62 protruding in a rear direction facing the rear frame part 22. The bent area 62 may be guided through the through-hole 221 of the rear frame part 22. The through-hole 221 may be slit-shaped. Adjacent the through-hole 221 of the rear frame part 22, the rear frame part 22 may comprise not further shown joint elements connectable to joint elements provided on the side frames 4 to engage with each other and forming the hinge joints 8. The bent area 62 may be arranged bent around the hinge joint 8, such as a commonly known hinge joint 8 from eyeglasses. The bent area 62 may be connected to the main electronic 54 and/or the battery 51 in the recess 43 of the side frame 4. In some designs, the electronic part 52 may comprise two laterally arranged bent areas 62. The rear frame part 22 may comprise two corresponding laterally arranged through-holes 221, each configured to receive one of the bent areas 62.

In some designs, the electronic part 52 and its bent area 62 may be configured reversible flexible and/or elastic. In some designs, the electronic part 52 and its bent area 62 may be configured stretchable.

The bent area 62 is configured to withstand a plurality of bending motions or movements between the respective side frame 4 and the front frame 2.

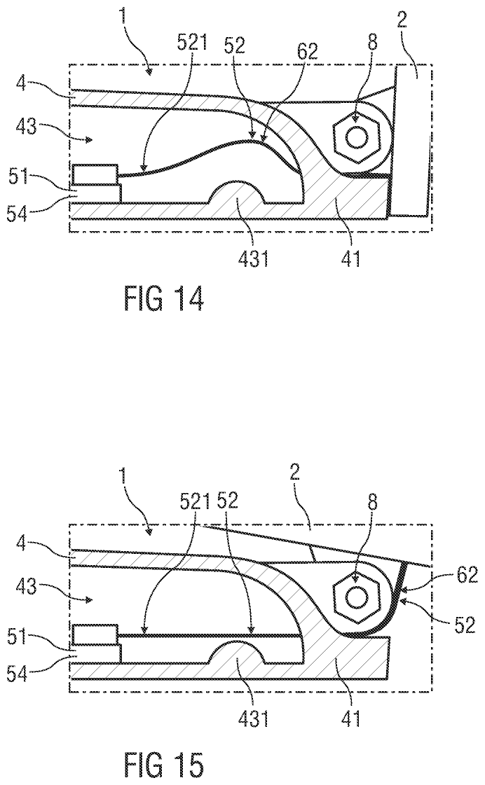

FIG. 14 shows schematically in cross section a top view of an embodiment of a portable light apparatus 1 in a use state, comprising a front frame 2, a pair of side frames 4 and an operating device 5 having a battery 51 and an electronic part 52 with a light device 53 and FIG. 15 shows schematically in cross section a perspective view of the portable light apparatus 1 in a non-use state. In particular, FIGS. 14 and 15 show a recess 43 of a side frame 4 housing the at least one battery 51 and optionally additionally a main electronic part 54.

A laterally bent area 62, such as a laterally arranged extension portion, of the electronic part 52 is configured to create a connection between the electronic part 52 carrying the light device 53 and the battery 51 and optionally additionally a main electronic part 54 arranged in the side frame 4. In particular, the electronic part 52 comprises the bent area 62 providing a connection area 521 to the main electronic part 54 and/or the battery 51. The connection area 521 may be connected to the main electronic part 54 and/or the battery 51 via at least one connector element, e.g. an electrical connector. The electronic part 52 may be always connected to the main electronic part 54 and/or the battery 51. The bent area 62 may be bent around the hinge joint 8. The bent area 62 may be configured in shape of a flat ribbon cable. The bent area 62 of the electronic part 52 may be arranged extending into an inside, in particular into the recess 43, of the side frame 4. The bent area 62 may be arranged bent around the hinge joint 8 as shown in FIG. 15. The bent area 62 may be connected to the main electronic 54 and/or the battery 51 in the recess 43 of the side frame 4.

In order to avoid undesired contact of the electronic part 52, in particular of its bent area 62, with inner wall portions of the recess 43 and/or inner side frame elements at least one bump element 431 is provided in the recess 43 which affects a movement of the bent area 62 when the portable light apparatus 1 is being moved from the use state, when worn by a user, to a non-use state, e.g. when stowed. Contact over a length of the bent area 62 when moving the side frame 4 relative to the front frame 2 may lead to undesired frictional wear and/or damage of the electronic part 52. Such a bump may direct the bent area 62 and/or the connection area 521 into a position of minimal friction, minimal mechanical resistance for the user and optimal use of the space provided by the recess 43.

The side frame 4 may comprise at least one bump element 431. The bump element 431 may comprise a rounded, curved and/or convex surface. The bump element 431 may be a stop element or an abutment. The bump element 431 may be molded or otherwise fixed in the recess 43 and/or formed from the same material as the side frame 4. The bump element 431 may be formed in one-piece with the side frame 43. The bump element 431 may protrude from an inner wall portion of the side frame 4 into the recess 43.

In the use state according to FIG. 14, the bent area 62 may undergo or experience less bending about the hinge joint 8 between the front frame 2 and the side frame 4 and therefore the electronic part 52, in particular its bent area 62 and/or the connection area 521, will be compressed in the recess 43. An unaffected compressed state of the connection area 521 in the recess 43 may lead to undesired shapes, bends and friction contacts of the connection area 521 and inner wall portions and/or inner side frame elements. Therefore, the bump element 431 may be configured such that it affects and/or forces the bent area 62 and/or the connection area 521 of the electronic part 52 to take a predefined shape and/or bend in a predefined way. In the use state, the bent area 62 and/or the connection area 521 may take a meandering shape. The bent area 62 and/or the connection area 521 may run without contact to inner sides of side frame walls through the recess 43. The bent area 62 and/or the connection area 521 may be bulbous and/or have a bulge at least in an area above the bump element 431.

In the non-use state according to FIG. 15, the bent area 62 may undergo or experience more bending compared to the use-state about the hinge joint 8 between the front frame 2 and the side frame 4 and therefore the connection area 521 of the electronic part 52 in the recess 43 will not be compressed. Instead, the bent area 62 and/or the connection area 521 may take a stretched state in the recess 43. The electronic part 52 may be permanently connected to the main electronic part 54 and/or the battery 51 in the use state and the non-use state. In the non-use state, the bent area 62 and/or the connection area 521 in the recess 43 may form a straight surface slightly above the bump element 431.

Due to the bump element 431, the bent area 62 and/or the connection area 521 can only move, e.g. deflect and escape, upwards with respect to the bump element 431 when the side frame 4 is folded out relative to the front frame 2. The bump element 431 is configured to generate a bulging and/or deflecting movement of the bent area 62 and/or the connection area 521.

In some designs, the bump element 431 may be arranged between the battery 51 and/or the main electronic part 54 and a front region 41 of the side frame 4 having a not further shown opening, e.g. a slit-shaped opening which corresponds to the through-hole 221 of the front frame 2, to guide the bent area 62 of the electronic part 52.

### LIST OF REFERENCES

1 portable light apparatus
1.1 communication unit
1.2 light therapy operation mode
2 front frame
21,22 frame part
221 through-hole
23 fastening device
231 fastening element
24 nose bridge
3 glasses (eyeglasses)
31 back surface
4 side frame
41 front region
42 rear region
43 recess
431 bump element
operating device
51 battery
52 electronic part, in particular auxiliary electronic part
521 connection area
522 opening
523 tab
5231 tab body
5232 first end
5233 second free end
5234 bent portion
53 light device
531 light emitting diode
5311 emission cone
5312 intersection area
54 electronic part, in particular main electronic part
541 processor module
542 radio module
543 light device driver
544 voltage regulation and battery charging circuitry
545 charging connector
546 power switch
61 to 63 bent area
71, 72 stiffener layer
8,81 hinge joint
9 charging case
91 housing
911,912 housing part
913 holding element
92 cavity
921 recess
93 charging operating device
931 charging port
932 charging electronics
94 magnet
200 control system
300 mobile device
301 communication unit
302 light therapy application
400 communication connection
500 central server
600 control and operating architecture
700 server-side
800 output device
900 streaming service
α1 to α4 angle
A1 arrow
C cornea
Ls line of sight
Ps, Pr, Pl plane
R1 radius
Vd vertex distance
Vp visual point
Vpa visual point area

What is claimed is:

1. A portable light apparatus configured to be worn by a user, the portable light apparatus comprising at least:
a front frame to house a pair of glasses and a pair of side frames connected to the front frame and
an operating device having at least a battery and at least one electronic part having a light device configured to emit light,
wherein the operating device is arranged in at least one of the side frames and/or the front frame,
wherein the light device comprises at least two light emitting diodes per each of the glasses and
wherein the light emitting diodes and/or their emission cones are arranged relative to one another such that the emission cones substantially intersect between a back surface of one of the respective glasses and an apex of the user's cornea to indirectly illuminate the line of sight of the user.

2. The portable light apparatus according to claim 1, wherein the light emitting diodes and/or the emission cones are arranged angled relative to one another such that an intersection area of the emission cones is substantially elliptical shaped.

3. The portable light apparatus according to claim 1, wherein the light emitting diodes and/or the emission cones are arranged aligned relative to one another such that an intersection area of their emission cones covers a length range between 5 mm and 35 mm between the back surface of the respective glasses and the apex of the user's cornea.

4. The portable light apparatus according to claim 1, wherein the light device comprises at least four light emitting diodes assigned to one, in particular to each, of the glasses.

5. The portable light apparatus according to claim 1, wherein the at least one electronic part is a flexible printed circuit board which has an outline shape of the front frame.

6. The portable light apparatus according to claim 1, wherein the at least one electronic part comprises two openings to receive the glasses, wherein the light emitting diodes assigned to one, in particular each, of the glasses are distributed around the openings.

7. The portable light apparatus according to claim 6, wherein the light emitting diodes are arranged on a plain surface of the at least one electronic part.

8. The portable light apparatus according to claim 6, wherein a number of projecting tabs are arranged in an area of the openings and each carrying one of the light emitting diodes.

9. The portable light apparatus according to claim 8, wherein the tabs are located in corner areas of each opening.

10. The portable light apparatus according to claim 8, wherein each tab comprises a tab body having a first end connected to the opening and a second free end extending away from the opening to which the light emitting diode is attached.

11. The portable light apparatus according to claim 10, wherein each tab has a bent portion arranged between the first end and the second free end.

12. The portable light apparatus according to claim 8, wherein each tab is bent about a bending radius smaller than 5 mm, in particular smaller than 4 mm.

13. The portable light apparatus according to claim 8, wherein each tab projecting towards a back surface of the at least one electronic part about an acute angle.

14. The portable light apparatus according to claim 8, wherein the at least one electronic part is made of one-piece and the tabs are shaped out from the opening.

15. The portable light apparatus according to claim 1, wherein the at least one electronic part comprises at least one bent area in a nose bridge area and a connection area to at least one of the side frames.

16. The portable light apparatus according to claim 1, wherein the operating device comprises at least one main electronic part, wherein the at least one main electronic part is a printed circuit board having an interface providing a connection to the at least one electronic part and an interface providing a connection to the battery.

17. The portable light apparatus according to claim 16, wherein the battery is configured to supply energy to the light device via the at least one electronic part and the at least one main electronic part comprises a number of interfaces to couple with an external charging device to recharge the battery and/or to couple with an external control device.

18. The portable light apparatus according to claim 1, wherein one of the side frames comprises a power switch element connected to the operating device.

19. The portable light apparatus according to claim 1, wherein the front frame comprises two frame parts permanently or temporarily connected to one another to hold the glasses and the at least one electronic part with its light device.

20. The portable light apparatus according to claim 19, wherein in a detached state of the front frame the glasses are exchangeable and wherein the at least one electronic part and the light device are held in one of the frame parts.

21. The portable light apparatus according to claim 1, wherein the operating device comprises a light device driver to control stroboscopic frequency and/or light sequences of the light device in the front frame.

22. The portable light apparatus according to claim 1, wherein the glasses are configured as regular transparent or colored glasses, ophthalmic grade glasses, color tinted glasses and/or functionally coated glasses and/or thermochromic coated glasses and/or electrochromic coated glasses.

23. The portable light apparatus according to claim 1, wherein the electronic device and the light device are configured to emit light with adjustable light color.

24. The portable light apparatus according to claim 23, wherein the light emitting diodes are configured to emit light with a wavelength between 380 nm and 800 nm and/or chosen from the group consisting of red, green, blue, yellow, orange, violet.

25. A control system of a portable light apparatus according to claim 1 and a mobile device, wherein the portable light apparatus comprises a communication unit, a light device and an electronic device, and the mobile device comprises another communication unit and a light therapy application,
wherein the light therapy application is configured to perform a communication connection via the communication units to the electronic device of the portable light apparatus from the mobile device to control at least one of the following light parameters: brightness, strobe frequency and light color of the light device.
